(19) United States
(12) Patent Application Publication
(10) Pub. No.: US 2009/0208975 A1
(43) Pub. Date: Aug. 20, 2009

(54) Device and methods for detecting a target cell

(75) Inventors: Sybil D'Costa, Miramar, FL (US); Robert S. Matson, Orange, CA (US); Alexander Burshteyn, Pembroke Pines, FL (US); Michael Simonian, Leguna Niguel, CA (US); Jinghai Wen, Miami, FL (US)

Correspondence Address: BECKMAN COULTER, INC.;Mitchell E. Alter, P.O. BOX 169015, MAIL CODE 32-A02, MIAMI, FL, 33116-9015, US

(73) Assignee: Beckman Coulter, Inc., Fullerton, CA (US)

(21) Appl. No.: 12/314,598

(22) Filed: Dec. 12, 2008

Related U.S. Application Data
(60) Provisional application No. 61/007,778, filed on Dec. 13, 2007.

(51) Int. Cl. G01N 33/567; C12M 1/34

(52) U.S. Cl. 435/7.2; 435/287.7; 435/287.2

(57) ABSTRACT

The present invention relates to devices for detecting intact target cells in a sample comprising a detection zone comprising an immobilized specific binding reagent, capable of forming a complex with a target analyte on a target cell. Once labeled, detection of the label indicates the absence, presence and/or amount of the target cell in a sample. The embodiments further relate to kits comprising the devices, and methods of using the devices to screen for the presence, absence, and/or amount of a target cell in a sample.

### CROSS-REFERENCE TO RELATED APPLICATIONS

This application claims priority to provisional application No. 61/007,778, filed on Dec. 13, 2007, which is hereby incorporated by reference in its entirety.

### BACKGROUND OF THE INVENTION

1. Field of the Invention

The present invention relates to devices, methods and kits for the semi-quantitative enumeration and detection of an analyte on a target cell using lateral flow and non-lateral flow assays.

2. Background

The Need for CD4 Testing of HIV Patients in Resource Limited Settings (e.g., Africa, South America, Caribbean, Eastern Europe, and Selected Countries in Asia)

Greater than 60% of the world's HIV infected population resides in Africa despite the fact that just over 12% of the world's population resides on the continent. An estimated 22 million people were living with HIV/AIDS in sub-Saharan Africa by the end of 2007. In that year, 1.5 million Africans died from AIDS. In the hardest hit countries the statistics are staggering: in Swaziland, 26.1% of the population was infected with HIV by the end of 2007. In Botswana, Lesotho, and South Africa, HIV infection rates are 23.9%, 23.2%, and 18.1%, respectively. The high cost of highly active antiretroviral therapy (HAART) has always been a barrier to AIDS treatment in third world nations, and despite the availability of new generic drugs and an improved health care system infrastructure, only 20% of adults who need HAART receive treatment.

In resource limited settings, like Africa, the current standard treatment protocol is to treat HIV infected individuals with HAART only after the disease has advanced to a stage where severe illness and infection has set in or when CD4 cell counts have dropped below 200 cells/μl. Recent studies suggest that over the lifetime of an HIV infected individual, the most cost-effective treatment protocol is to monitor CD4 cell counts and initiate HAART treatment when cell counts fall below a threshold limit, usually 200 or 350 cells/μl. Thus, treatment is initiated before the immune system is severely compromised. Individuals started on drug therapy before symptoms appear suffer from fewer opportunistic infections and require fewer costly hospitalizations. Additionally, HIV infected individuals who are treated with HAART before the onset of symptoms but after CD4 cell counts have dropped below a threshold of 200-350 cells/μl have an increased life expectancy of 7 to 12 months and are less likely to pass the infection on to others.

Despite the clear advantages of monitoring CD4 cell count in HIV infected individuals, such testing is not frequently done in resource limited settings. One barrier to CD4 testing is the high start up costs. The flow cytometers that are used to perform blood analysis for CD4 can cost up to $150,000, are complex to maintain, and expensive to operate. In addition, these instruments need highly trained laboratory personnel to run the CD4 enumeration assays as well as trained phlebotomists to obtain the venous blood for CD4 testing. Another major obstacle is simply getting blood samples to a lab for analysis. In rural areas, shipping blood samples to centralized laboratories for CD4 testing in a timely fashion, preferably within 24 and at the maximum 48 hrs after blood draw is also problematic (Note, the blood does not need to be refrigerated). Thus, there is a need for an inexpensive, easy to operate, rapid point-of care test to determine CD4 cell counts in HIV infected individuals. The present invention addresses this and other related needs by disclosing a lateral flow immunoassay that can semi-quantitatively detect target cells, such as CD4+ T-cells, in a sample, such as whole blood. The test is rapid, providing results within a few minutes, easy to administer, and requires no storing or shipping of blood samples such that the decision to treat or not treat a patient can be made on site. The components of the test kit are also stable at elevated temperatures so that it can be transported and used in remote regions without the need for cold chain storage. Finally, in certain embodiments of the present invention, the test requires no machinery or electricity to operate; the read out is a simple band or bands that can be visualized with the naked eye.

Lateral Flow Immunoassays

Lateral flow immunoassays utilizing sorbent materials are widely used in many different areas of analytical chemistry and medicine. Such assays enable relatively sophisticated chemical analysis to be quickly performed by unskilled users upon complex samples, such as urine, blood, and environmental samples, and typically return results within a few minutes using minimal amounts of additional instrumentation.

Generally, lateral flow immunoassays are composed of several different types of membrane material pressed together. Typically a liquid sample will be applied to a separation membrane. This membrane separates the liquid portions of the sample from solid particles, such as red blood cells. The fluid portion of the sample then travels through a wicking membrane, which transports the fluid by capillary action, to a conjugate release membrane, which stores an antibody reactive with the analyte of interest present in the liquid sample. The antibody in turn is usually labeled with small light absorbing particles, such as colloidal gold particles. The antibody and colloidal gold particles will typically be stored in a dry state, and be rehydrated by the fluid sample.

As the fluid sample passes by the conjugate release membrane, the antibody and detection particles are exposed to the fluid, and the antibody binds to its binding site (epitope) on the analyte molecules of interest that are present in the sample. The fluid then passes into a reaction membrane. Typically the reaction membrane is made from a thin layer of material, such as nitrocellulose, that is capable of binding protein in a non-covalent fashion. A second capture antibody, capable of binding to a different epitope on the analyte molecule, is applied to the membrane, usually in the form of a thin line that provides good visual contrast for subsequent visual assessment. The capture antibody binds tightly to the membrane, and remaining membrane protein binding capability is then removed by incubation with excess “blocking protein”. As the fluid passes through the reaction membrane, a final absorbent membrane that is in contact with the reaction membrane removes excess fluid. Analyte molecules in the sample bind to the capture antibody, which in turn is bound to the reaction membrane. The detection antibody in turn also binds to the analyte molecules bound on the reaction membrane, and the colored detector particles bind to the detection antibody, forming a sandwich that produces a visible signal when analyte is present.

Previous lateral flow immunoassay work is exemplified by U.S. patents and patent application publications: U.S. Pat. Nos. 5,602,040; 5,622,871; 5,656,503; 6,187,598; 6,228,660; 6,818,455; 2001/0008774; 2005/0244986; U.S. Pat. No. 6,352,862; 2003/0207465; 2003/0143755; 2003/0219908; 2006/0240569, U.S. Pat. Nos. 5,714,389; 5,989,921; 6,485,982; 2006/0040405; 5,656,448; 5,559,041; 5,252,496; 5,728,587; 6,027,943; 6,506,612; 6,541,277; 6,737,277 B1; 5,073,484; 5,654,162; 6,020,147; 4,956,302; 5,120,643; 6,534,320; 4,942,522; 4,703,017; 4,743,560; 5,591,645; and RE 38,430.

Other types of diagnostic assays have also been developed that use chromatographic principles. These are exemplified by hand-held cholesterol assays, such as the work disclosed in U.S. Pat. Nos. 4,999,287; 4,987,085; 4,959,324; 5,204,063, and 5,508,664, which discuss a number of ways in which small buffer packages can be packaged in a single hand-held diagnostic unit.

There is a need for improved analytical technology to provide rapid, easy-to-use and semi-quantitative techniques to detect intact cells in biological samples, to provide a relatively immediate result without the need to use additional equipment to visualize the signal, and to overcome the lack of quantitation and poor sensitivity of traditional lateral flow immunoassays, as well as overcoming the lack of reproducibility of the pore structure of traditional lateral flow assay membranes. The present invention addresses this and other related needs.

### BRIEF SUMMARY OF THE INVENTION

In one aspect, the present invention provides devices for detecting an analyte on an intact target cell in a sample.

In one aspect, the present invention provides devices for detecting an analyte in a sample. The device for detecting a target cell in a sample can comprise one or more non-porous support materials capable of generating lateral flow of intact cells. The non-porous support materials comprise a sample receiving zone for receiving a sample comprising a target cell, and one or more detection zones comprising an immobilized specific binding reagent capable of forming a complex with a first target analyte on the target cell, wherein the immobilized specific binding reagent is present in an amount sufficient to form a complex with the target cell at a specified level. The sample receiving zone and at least one detection zone are typically arranged in the one or more support materials such that the sample is capable of lateral flow sequentially across the sample zone and to the detection zone. The device can further comprise a control zone comprising an immobilized control specific binding reagent.

In another aspect, the invention provides a device for detecting a target cell, comprising one or more support materials comprising a detection zone comprising an immobilized specific binding reagent capable of forming a complex with a first target analyte on a target cell. The immobilized specific binding reagent is present in an amount sufficient to form a complex with the target cell at a specified level. The device can further comprise a control zone comprising an immobilized control specific binding reagent.

In another aspect, in the device for detecting a target cell, the one or more non-porous support materials is comprised of a plurality of projections substantially perpendicular to the surface of the support material. These projections have a height, a diameter, and spacing between the projections sufficient to generate lateral flow of a sample comprising intact cells across the support material.

The invention further provides methods of using the devices described herein in methods to detect target cells in a sample. In one aspect, the method for detecting a target cell in a sample comprises providing a device comprising one or more non-porous support materials capable of generating lateral flow, the one or more non-porous support materials comprising a sample receiving zone for receiving a sample, and one or more detection zones comprising an immobilized specific binding reagent capable of forming a complex with a first target analyte on the target cell, if present in the sample. The method further comprises contacting the sample receiving zone with a sample containing or suspected of containing a target cell, transporting the sample to the detection zone, wherein the immobilized specific binding reagent forms a complex with the target cell, if present in the sample, and detecting the target cell. The target cell is detectable due to the presence of a label. The target cell can be labeled before the sample is applied to the sample receiving zone, before the target cell reaches a detection zone, and/or after the target cell reaches a detection zone. The detection of the label indicates the absence, presence and/or amount of the target cell. In a further embodiment, the sample is transported to a control zone comprising an immobilized control specific binding reagent, wherein the immobilized control specific binding reagent forms a detectable complex with one or more control cells. The detection of a control cell in the control zone indicates the validity of the test results.

In a further aspect, the method for detecting a target cell in a sample comprises providing a device comprising a support material comprising a detection zone comprising an immobilized specific binding reagent capable of forming a complex with a target analyte on the target cell, if present in the sample, contacting the detection zone with a sample containing or suspected of containing a target cell, wherein the immobilized specific binding reagent forms a complex with the target cell, and detecting the target cell. The target cell is detectable due to the presence of a label. The target cell can be labeled before and/or after the sample is applied to the sample receiving zone. The detection of the label indicates the absence, presence and/or amount of the target cell.

In a further aspect, the method of using the devices described herein to detect target cells in a sample comprises providing a device comprising a support material comprising a plurality of projections substantially perpendicular to the surface of the support material. These projections have a height, a diameter, and spacing between the projections sufficient to generate lateral flow of a sample comprising intact cells across the support material.

In a particular aspect, the devices described herein are used in methods to detect CD4+ T-cells in a whole blood sample. In this aspect, the target cell is a CD4+ lymphocyte, the analyte is CD4, and the sample is whole blood. Also in this aspect, the detection zone comprises an immobilized specific binding reagent that forms a complex with CD4. One or more detection zones can be present. In a further aspect, the detection of CD4+ cells in a particular detection zone provides a semi-quantitative assessment of CD4+ T-cell count in the sample.

### BRIEF DESCRIPTION OF THE DRAWINGS/FIGURES

The patent or application file contains at least one drawing executed in color. Copies of this patent or patent application publication with color drawings will be provided by the Office upon request and payment of the necessary fee.

FIG. 1 shows an exemplary illustration of a close-up view of a portion of a micropillar region of the support material of the present invention

FIG. 2 shows an overview of a method of using a device constructed according to one embodiment of the present invention.

FIG. 3 shows a schematic of a device constructed according to one embodiment of the present invention.

FIG. 4 shows an overview of a method of using a device constructed according to one embodiment of the present invention.

FIG. 5 shows a schematic of a device constructed according to one embodiment of the present invention.

FIG. 6 shows an overview of a method of using a device constructed according to one embodiment of the present invention.

FIG. 7 shows a schematic of a device constructed according to one embodiment of the present invention.

FIG. 8 shows an overview of a method of using a device constructed according to one embodiment of the present invention.

FIG. 9 shows a schematic of a device constructed according to one embodiment of the present invention.

FIG. 10 shows an exemplary device constructed according to one embodiment of the present invention.

FIGS. 11A-C (in color) show fluorescent data obtained from an embodiment of a lateral flow device of the invention.

FIGS. 12A-F show microscopic images obtained from an embodiment of a lateral flow device of the invention.

FIGS. 13A-E (in color) show fluorescent data obtained from several embodiments of a lateral flow device of the invention where the pH of the capture antibody solution was varied.

FIG. 14 shows quantification of cell capture on a lateral flow device of the invention.

FIGS. 15 A-B (in color) show fluorescent data obtained from several embodiments of a lateral flow device of the invention where cell number and capture antibody was varied.

FIGS. 16 A-D show microscopic images obtained from an embodiment of a lateral flow device of the invention using different capture antibodies.

FIG. 17 shows the cross-inhibition of unlabeled CD4+ T cells to labeled CD4+ T-cell binding to the detection zone on a lateral flow device of the invention indicating specificity of binding.

FIG. 18 shows the cross-inhibition capabilities of monocytes (which also express CD4) to CD4+ T-cell binding to the detection zone on a lateral flow device of the invention.

FIG. 19 shows the cross-inhibition of soluble recombinant CD4 protein to CD4+ T-cell binding to the detection zone on a lateral flow device of the invention indicating specificity of binding.

FIG. 20 A-B show the testing of CD3 labeled black beads as a detector in the lateral flow device of the invention.

FIGS. 21A-B show that the viability and phenotypic characteristics of cells are maintained after lateral flow on the device of the invention.

FIG. 22 illustrates the steps involved in performing an assay of the invention when whole blood samples are depleted of monocytes before application to the device.

FIG. 23 shows the efficiency of monocyte depletion of whole blood as analyzed on a Coulter® LH 750 Hematology Analyzer.

FIGS. 24 A-B show the efficiency of monocyte depletion of whole blood as analyzed by flow cytometry.

FIG. 25 shows the effect of monocyte depletion on CD4+CD3+ cell counts in a whole blood sample.

FIG. 26 shows the evaluation of normal donor whole blood samples depleted of monocytes after lateral flow on a device of the invention.

FIG. 27 A-B demonstrates the ability of a device of the invention to differentiate between CD4+ T-cell counts of 250 cells/μl versus 350 cells/μl.

FIG. 28 shows an evaluation of intra- and inter-operator variability in operating the device of the invention.

FIG. 29 shows the evaluation of whole blood samples from HIV infected individuals with known CD4+ T-cell counts on a device of the invention.

FIG. 30 shows the evaluation of whole blood samples from HIV infected individuals with known CD4+ T-cell counts on a device of the invention.

FIG. 31 demonstrates the feasibility of on-board monocyte depletion when a monocyte depletion zone is incorporated into a device of the invention.

### DETAILED DESCRIPTION OF THE INVENTION

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

The term “about” when used in the context of numeric values denotes an interval of accuracy, familiar and acceptable to a person skilled in the art. In at least some embodiments of the present invention, about means a value±10% of the indicated number.

As used herein, the term “sample” refers to anything which can contain a target cell for which an analyte assay is desired. The sample can be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). In one embodiment of the invention, the sample is whole blood. The whole blood can be obtained from a finger-prick or other non-venous method, or can be obtained from a venous source.

As used herein, the term “target cell” refers to an intact cell for which an analyte assay is desired in order to detect the presence and/or determine the amount of the target cell in the sample. Target cells can include whole cells, i.e., live cells, as well as non-living cells such as ghosted cells and permeabilized nonviable cells whose cell membranes remain intact. In one aspect, the target cell is an intact cell. The target cell will have at least one analyte that is specifically bound by a binding reagent, such as an antibody. The sample can be examined directly or can be pretreated to render the analyte on the target cell more readily detectable. Typically, the target cell of interest is determined by detecting an agent probative of the target cell such as specific binding of a binding reagent coupled to a label, wherein the binding reagent is specific to an analyte on the target cell. The presence of the label will be detected only when the target cell is present in a sample. Thus, the agent (binding reagent coupled to a label) probative of the analyte on the target cell becomes the analyte that is detected in an assay. In one embodiment of the invention, the target cell is a CD4+ lymphocyte. In a further aspect, the target analyte of the CD4 lymphocyte is a CD3 or CD4 antigen.

As used herein, “antibody” is used in the broadest sense. Therefore, an “antibody” can be naturally occurring or man-made such as monoclonal antibodies produced by conventional hybridoma technology and/or a functional fragment thereof. Antibodies of the present invention comprise monoclonal and polyclonal antibodies as well as fragments (such as Fab, Fab′, F(ab′)2, Fv) containing the antigen-binding domain and/or one or more complementarity determining regions of these antibodies.

As used herein, “monoclonal antibody” refers to an antibody obtained from a population of substantially homogeneous antibodies, i.e., the antibodies comprising the population are identical except for possible naturally occurring mutations that are present in minor amounts. As used herein, a “monoclonal antibody” further refers to functional fragments of monoclonal antibodies.

As used herein, the term “specifically binds” refers to the specificity of a reagent (such as an antibody) such that it preferentially binds to a defined target. A reagent “specifically binds” to a target if it binds with a greater degree of affinity, greater avidity, more readily, and/or with greater duration than it binds to other substances. For example, an antibody specific for a certain analyte will have an affinity for its analyte that is at least about 10-fold, at least about 100-fold, at least about 1000-fold, at least about 10,000-fold or higher than for other substances. For example, for IgG antibodies, the K_{dissociation} will range from 10⁻⁷-10⁻¹¹ M. Recognition by an antibody of a particular target in the presence of other potential targets is one characteristic of such binding. In one embodiment, the specific binding reagent can distinguish the analyte from other substances that are present or likely to be present in the sample to be tested. Specific binding reagents include, but are not limited to antibodies, antigens, haptens, biotin, avidin, lectin, sugar, nucleic acids, receptors and their ligands.

As used herein, the term “support material” refers to any substance capable of immobilizing a specific binding reagent. The support material can be porous or non-porous. The porous and/or non-porous support materials are further capable of providing lateral flow. Non-limiting examples of non-porous support materials include plastic substrates, glass substrates, metal substrates and/or silicon substrates. Such substrates can be layered upon each other, and/or layered with porous substrates. The term “substrate” means the carrier or matrix to which a sample is added, and on or in which the determination is performed, or where the reaction between analyte and specific binding reagent takes place.

The support material can be treated to provide a chemically reactive group on the surface of the support material. This chemically reactive group can be useful in immobilizing one or more specific binding reagents, and includes all organic and inorganic groups used in covalent coupling of molecules to solid surfaces and known to persons skilled in the art, such a hydroxyl, carboxyl, amino, sulphonate, thiol, and aldehyde groups, etc.

In further embodiments, the surface of the support material is coated or derivatized, e.g. using techniques such as sputtering, vapor deposition and the like, and given a coating of silicon, a metal or other. In one embodiment the substrate is given a hydrophilic treatment or coating, e.g. by subjecting the substrate to an oxidative treatment, such as by gas plasma treatment, coating with a hydrophilic substance such as silicon oxide, hydrophilic polymers such as dextran, polyethylene glycol, heparin and derivatives thereof, detergents, biologic substances such as polymers, etc. In one embodiment, the coating or derivatization itself is a chemically reactive group.

The terms “zone,” “area,” “location” and “site” are used interchangeably in the context of this description, examples and claims to define parts of the fluid passage on a substrate, either in prior art devices or in a device according to an embodiment of the invention. In the context of devices that do not require fluid passage, the term is used to define a region of the device comprising an immobilized specific binding reagent. The various zones, including the wash zone, sample receiving zone, detection zone, depletion zone and/or the control zone, can take any suitable form. The locations can be dots, circles, squares, zones or lines, etc. In one example, one or more detection zones and/or the control zone are in the form of a line or lines. In one aspect, the zone(s) extends across the width of the support material.

As used herein, the term “sample receiving zone” refers to the portion of the device that is contacted with the sample comprising or suspected of comprising the target cell.

As used herein, the term “detection zone” refers to one or more portions of the device that comprises an immobilized specific binding reagent capable of forming a complex with a first target analyte on a target cell. In one aspect, the immobilized specific binding reagent is present in an amount sufficient to form a complex with the target cell at a specified level, such that detection of the target analyte within the detection zone provides an indication of the presence and/or amount of the target cell. It is contemplated that the devices can comprise one or more detection zones. Each detection zone can comprise the same or a different immobilized specific binding reagent.

As used herein, the term “immobilization” refers to the attachment or entrapment, either chemically or otherwise, of a binding reagent to one or more support materials in a manner that restricts the movement of the binding reagent. A binding reagent can be immobilized on the support material by any suitable methods. For example, binding reagent can be immobilized by absorption, adsorption, or covalent binding to the support material, or by attaching to another substance or particle that is immobilized to the desired location on the support material. In one aspect of the invention, the binding reagent is covalently attached directly or indirectly to the support material.

As used herein, a “control zone” refers to one or more portions of the device that comprises a control specific binding reagent—a binding reagent that binds specifically to an analyte on one or more control cells. In one aspect, the control zone is downstream from, but in fluid communication with one or more detection zones. As such, detection of control cells or analytes in the control zone indicates a valid test result.

As used herein, a “label” is a substance that is capable of producing a detectable signal. It is typically coupled to a binding reagent specific for an analyte on a target cell. When the labeled binding reagent couples to the target cell, detection of the label provides an indication of the presence and/or amount of the target cell. The labeled binding reagent can couple to a target cell either before or after the target cell forms a complex with the immobilized specific binding reagent in a detection zone of the devices of the invention. Labels usually do not change or affect the underlining assay process. Labels include, but are not limited to, a radio-active material, a magnetic material, a quantum dot, an enzyme, a liposome-based label, a chromophore, a fluorophore, a dye, a nanoparticle, a quantum well, a composite organic-inorganic nano-cluster, a colloidal metal particle, latex particles, or combinations thereof. In one aspect, the label comprises a colloidal gold particle, a colloidal silver particle conjugate, a latex particle, and/or a liposome-based particle. In certain aspects, the label is visible without the use of other instrumentation other than the possible use of a magnifier. In one embodiment, the intensity of the label in a detection zone indicates the number of target cells in the sample. Detection of the label indicates the presence and/or amount of the target cell.

As used herein, “mammal” refers to any of the mammalian class of species, preferably human (including humans, human subjects, or human patients). Mammals include, but are not limited to, farm animals, sport animals, pets, primates, horses, dogs, cats, mice, and rats.

As used herein, “treatment” means any manner in which the symptoms of a condition, disorder or disease are ameliorated or otherwise beneficially altered. Treatment also encompasses any pharmaceutical use of the compositions herein.

As used herein, “disease or disorder” refers to a pathological condition in an organism resulting from, e.g., infection or genetic defect, and characterized by identifiable symptoms. In one aspect, the disease is infection with HIV.

As used herein, the term “subject” is not limited to a specific species or sample type. For example, the term “subject” can refer to a patient, and frequently a human patient. However, this term is not limited to humans and thus encompasses a variety of mammalian species.

As used herein, “afflicted” as it relates to a disease or disorder refers to a subject having or directly affected by the designated disease or disorder.

As used herein, the term “lateral flow” describes the movement of a liquid sample along a solid substrate via capillary action. Capillary action is the ability of a substance (usually a solid material) to draw another substance (usually a liquid) into or across it. It occurs when the adhesive intermolecular forces between the liquid and the solid substance are stronger than the cohesive intermolecular forces inside the liquid.

### B. Devices and Methods for Detecting an Analyte in a Sample

The present devices and methods can be used to detect an analyte on a target cell in any suitable sample.

In another aspect, the present invention provides devices for detecting an analyte in a sample. The device for detecting a target cell in a sample can comprise one or more non-porous support materials capable of generating lateral flow of intact cells. The non-porous support material(s) comprise a sample receiving zone for receiving a sample comprising a target cell, and one or more detection zones comprising an immobilized specific binding reagent capable of forming a complex with a first target analyte on the target cell, wherein the immobilized specific binding reagent is present in an amount sufficient to form a complex with the target cell at a specified level. The sample receiving zone and at least one detection zone are typically arranged in the one or more support materials such that the sample is capable of lateral flow sequentially across the sample zone and to the detection zone. The device can further comprise a control zone comprising an immobilized control specific binding reagent.

The non-porous support material can comprise a plastic substrate, a glass substrate, and/or a silicon substrate, metal, polystyrene, or polypropylene. These materials can be chemically activated to enable covalent coupling of immobilized reagent to the non-porous support. In one aspect, the plastic substrate comprises a cyclo-olefin polymer substrate.

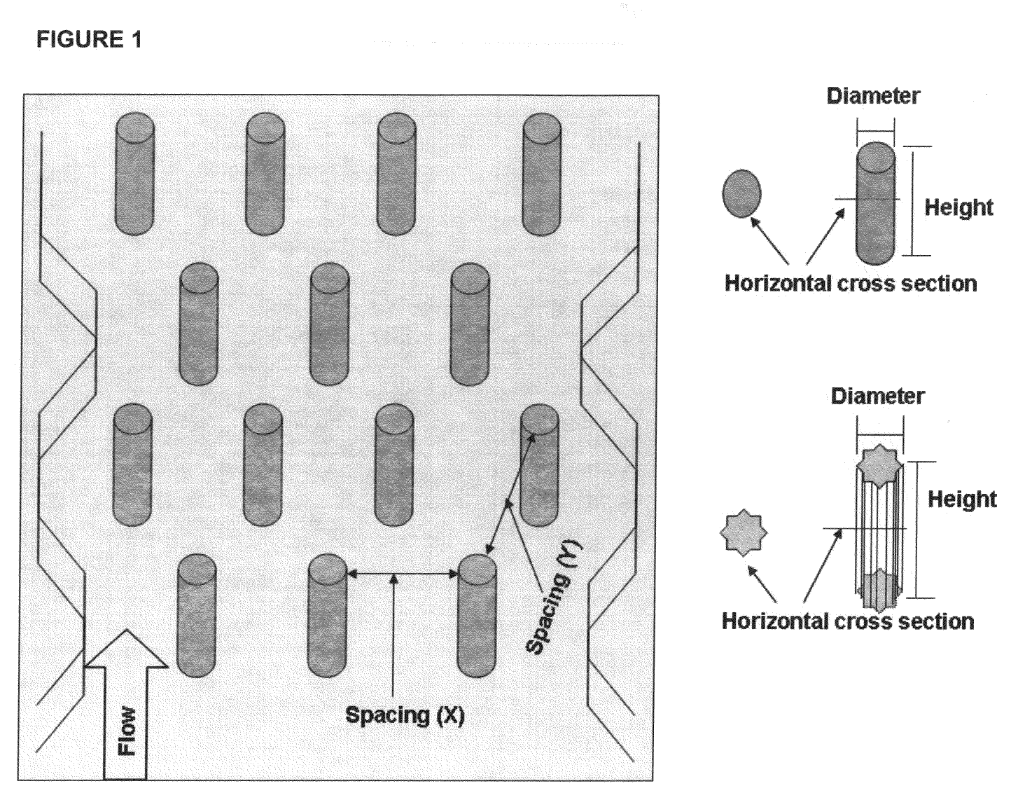

The non-porous support material(s) of the invention can comprise a plurality of micropillars protruding upwards from the substantially horizontal surface of the material, as generally described in U.S. Patent Publication Nos. 2005/0042766, 2006/0285996 and/or 2007/0266777, the micropillars described in these publications are herein incorporated by reference. An exemplary illustration of a close-up view of a portion of a micropillar region of the support material of the present invention is provided in FIG. 1 of the present application.

In a further aspect, the micropillars are spaced sufficiently to permit lateral flow of a whole blood cell between the micropillars. For example, monocytes range in size from 10 to 20 μm. Thus, to allow lateral flow of a whole blood sample containing monoctyes, micropillars should be spaced at least about 20 μm apart. The micropillars can be spaced about 20 μm to about 200 μm or more apart, measuring from the edge of one micropillar to the edge of the adjacent micropillar. In a specific embodiment, the micropillars are spaced about 20 μm to about 50 μm apart. In another specific embodiment, the micropillars are spaced about 27 μm to about 33 μm apart. In a preferred embodiment, the micropillars are spaced about 30 μm apart. In one aspect, the micropillars are evenly spaced. In another aspect, the micropillars are not evenly spaced. The spacing between micropillars can be described in two dimensions—the space between adjacent micropillars in the X dimension (sideways across the width of the support material) or in Y dimension (front to back along the length of the support material). See FIG. 1 for an illustration of the micropillars and the spacing between the micropillars. The micropillars can have a spacing in the X dimension that is different from the spacing in the Y dimension. In another embodiment, different zones along the length of the support material in a single device can have different spacing of micropillars. For example, the spacing of micropillars in the sample receiving zone can be 50 μm and the spacing of micropillars in the detection zone can be 30 μm. The flow of a liquid sample along the support material by capillary action is controlled by the interaction of the liquid with the surface of the support material, including the support material itself as well as any hydrophilic coating or wetting agents applied to the support material. Thus, changing the spacing of the micropillars will change the interactions between the liquid and the support, and necessarily change the speed at which the liquid sample flows. A skilled user can adjust the flow of the sample across the device as a whole, or in specific zones of the device, by changing the spacing of micropillars. In general, more closely spacing the micropillars will increase the surface area per unit area of support material and thus will tend to affect the speed at which a liquid sample flows.

In another aspect, the micropillars have a maximum diameter of about 10 μm to about 160 μm. In a specific embodiment, the micropillars have a maximum diameter of about 20 μm to about 60 μm. In another specific embodiment, the micropillars have a maximum diameter of about 45 μm to about 55 μm. In a preferred embodiment, the micropillars have a maximum diameter of about 50 μm.

In a further aspect, the micropillars have a height of about 50 μm to about 150 μm. In most embodiments of the present invention, the micropillars are of a sufficient height that the volume of sample applied to the device flows between the micropillars and not over them. The immobilized specific binding reagent is associated with the surface of the support material and the surfaces of the micropillars. In embodiments of the present invention where semi-quantitative detection of a target cell is desired, it is important that the sample flows between the micropillars and not over them so that all of the target cells in the sample have a chance to interact with the immobilized binding reagent. Any target cells in a sample that flow over the micropillars will not be captured and any determination of cell number will be inaccurate. In another specific embodiment, the micropillars have a height of about 58 μm to about 72 μm. In a preferred embodiment, the micropillars have a height of about 65 μm. In another preferred embodiment, the micropillars have a height of 130 μm.

The micropillars of the invention can have a horizontal cross section of any shape. In one embodiment, the cross-section of the micropillars is oval or circular in shape. In another embodiment, the cross-section of the micropillars is star shaped. In another embodiment, the cross-section of the micropillars is rectangular in shape. In another embodiment, the cross-section of the micropillars is elliptical or egg-shaped.

Increasing the number of micropillars on the support material will increase the surface area available for immobilizing a specific binding reagent, and thus will increase the capacity of the device to bind a target cell of interest. Furthermore, increasing the height of the micropillars, or changing their shape (such as from a circular shape to a star shape or rectangular shape or oval shape) will also affect the available surface area. By increasing the available surface area and by increasing the micropillar density per unit area, an operator can increase the sensitivity of the assay. Thus, to the extent that the operator knows the capacity for cell capture required for any given assay, one can design a micropillar configuration by adjusting the spacing, height, width, diameter and/or shape of the micropillars, as well as adjusting the titration of the immobilized specific binding reagent, to satisfy the requirement. In this regard, the present invention provides a flexible platform for designing cell capture immunoassays.

In a further aspect, the device is capable of generating lateral flow of ˜5 to ˜200 μL of a sample. In one embodiment, the device is capable of generating lateral flow of ˜10 μL to ˜30 μL, ˜50 μL, ˜75 μL, ˜100 μL, ˜125 μL, ˜150 μL, ˜175 μL and/or ˜200 μL of a sample. By increasing the heights of the micropillars, changing the shapes, and/or adding a sample reservoir in the sample receiving zone, a skilled user can vary the volume of sample that the device is capable of flowing. A micropillar configuration with heights of 65 μm, diameters of 50 μm and spacing between the micropillars of 30 μm in both the X and Y directions is capable of flowing a sample volume of ˜10 μL to ˜30 μL, although other configurations are possible and can be determined by a skilled user. In another embodiment, the micropillars of the device have a height, diameter, and spacing to provide lateral flow of a sample at a speed that allows for optimal binding of the target analyte on the target cell to the immobilized binding reagent. The speed of lateral flow of a liquid sample by capillary action across the surface of the support material is dictated by the attractive forces between the liquid and the support material surface. Slowing down the flow gives the target cells more time and more opportunity to encounter and bind to an immobilized specific binding reagent. Increasing the support material surface area increases the attractive forces; conversely, decreasing the support material surface area decreases the attractive forces. Thus, the user can increase or decrease the rate of flow by adjusting the micropillar configuration (dimensions and/or shape) to suit the needs of a particular application. In a specific embodiment, the micropillars have a diameter of about 50 μm, a height of about 65 μm, and a spacing of about 30 μm. In another specific embodiment, the micropillars have a diameter of about 30 μm, a height of about 130 μm, and a spacing of about 30 μm. In another specific embodiment, the micropillars have a diameter of about 30 μm, a height of about 65 μm, and a spacing of about 30 μm. In another specific embodiment, each of the above configurations have a reservoir in the sample receiving zone which allows for increased volume of the sample to be added while still enabling the sample added to flow between but not over the micropillars.

In one aspect, the present invention provides devices for detecting an analyte in a sample. The device for detecting a target cell in a sample can comprise one or more porous support materials. The porous support materials are capable of generating lateral flow of intact cells or are capable of allowing radial diffusion of a sample comprising intact cells. The porous support material(s) comprise a sample receiving zone for receiving a sample comprising a target cell, and one or more detection zones comprising an immobilized specific binding reagent capable of forming a complex with a first target analyte on the target cell, wherein the immobilized specific binding reagent is present in an amount sufficient to form a complex with the target cell at a specified level. The sample receiving zone and at least one detection zone are typically arranged in the one or more support materials such that the sample is capable of lateral flow or radial diffusion sequentially across the sample zone and to the detection zone. The device can further comprise a control zone comprising an immobilized control specific binding reagent.

The porous support material can comprise plastic, silicon, cellulose ester, nylon, particulate silica, polyethylene, polystyrene, polyvinyl, polypropylene, polyacrylonitrile, DEAE, polyamide, polyacrylamide, cellulose agarose, dextran, nitrocellulose, cellulose acetate, PES (polyethersulfone), glass fiber membranes or mixtures thereof. Other appropriate porous materials can also be used. A skilled artisan can choose a porous support material appropriate for the desired use. Variables to consider when selecting a porous support material for a lateral flow assay or a radial diffusion assay include the porosity of the material, the pore size, the flow rate of the material, the capacity of the material, and the binding capacity. Generally, support materials useful in the methods of the present invention have a hydrophobic backbone with hydrophilic surface, a surface neutral charge, low non-specific binding, consistent pore size, thickness and protein binding capacity, optimal porosity enabling radial diffusion or non-lateral flow, low coefficient of variation (CV) for capillary rise time over shelf life, are amenable to chemical modification to enable covalent conjugation to a specific binding reagent, and have multiple functionality-conjugate application, such as sample application, reaction surface, and wicking action.

Membrane porosity describes the fraction of the membrane that is air (e.g. a membrane with a porosity of 0.7 is 70% air), and will have an impact on the flow rate of the membrane.

As is well known to those in the art, the pore size of a porous material can be determined by hard particle challenge testing i.e., by determining the maximum diameter of spherical particles which can pass through the material. Alternatively, the pore size of a fibrous material may be determined by measuring its ‘bubble point’. The bubble point is the pressure required to force air through a material wet with water, and correlates with the pore size as measured by particle retention (although at extremes of pressure and pore size, the correlation may be weaker). Measurement of the flow rate through a material can also be used to determine pore size. The sample used in the assay of the present invention comprises intact cells that range in size up to 20 μm. Thus, it is desirable to select a porous support material with a pore size of at least about 20 μm to allow the cells to flow through.

The flow rate of a material is determined empirically, and will vary according to the viscosity of the sample used. Data for the flow rates of specific materials with specific sample types are supplied by the manufacturer.

The capacity of a material is the volume of sample that can pass through a given material per unit time, and is determined as a factor of the length (L), width (W), thickness (T), and porosity (P) of the material: L×W×T×P=capacity.

The binding capacity of a material is the amount of a protein that will bind per unit area. This information can often be obtained from the manufacturer of the material. A second important calculation is the determination of the amount of antibody that can be bound per unit area of the support material (pertaining to the detection zone, control zone, and depletion zone). This calculation is a factor of the binding capacity of the material and the area of the particular zone.

The porous support materials can be supported by other materials to provide strength and durability. For example, the support materials can be backed by non-woven spun fabric such as Hollytex® (Ahlstrom Filtration Co.) or Whatman paper filter.

In another aspect, the invention provides a device for detecting a target cell, comprising one or more support materials comprising a detection zone comprising an immobilized specific binding reagent capable of forming a complex with a first target analyte on a target cell. The immobilized specific binding reagent is present in an amount sufficient to form a complex with the target cell at a specified level. The device can further comprise a control zone comprising an immobilized control specific binding reagent.

The device can comprise a suitable support, e.g., the support material can be supported by a solid backing. Any suitable solid backing can be used. For example, the solid backing can be plastic, glass, metal, polystyrene, polypropylene, or silicon. The device can comprise a housing that covers at least the detection zone on the support material, wherein the housing comprises a sample application port to allow sample application to the sample receiving zone that is upstream from the detection zone and an optic opening around the detection zone and/or control zone, if present, to allow detection of a label at the detection zone and/or control zone. The device can also comprise a port near the wash zone to allow the addition of a washing solution to the device and/or a port near the waste zone to allow the placement of a wick on the device. In a further embodiment, the device can comprise a housing that covers a portion of the support material, wherein the housing comprises a sample application port to allow sample application to the support material and an optic opening around the detection zone to allow detection of a label at the detection zone. In this embodiment, the sample application port and optic opening can be the same structure. The housing can be made of any suitable material. For example, the housing comprises a plastic material. In certain embodiments, the housing acts as an evaporative barrier, keeping the device in a humidified environment.

In another embodiment, an absorptive material can be put inside the housing for the purpose of holding moisture and creating a humidified environment around the support material. Absorptive materials can comprise filter paper, sponges, cellulose based materials such as cotton, wool, other natural and synthetic textiles, or any other material that can hold moisture.

The invention further provides methods of using the devices described herein in methods to detect target cells in a sample. In one aspect, the method for detecting a target cell in a sample comprises providing a device comprising one or more non-porous support materials capable of providing lateral flow, the one or more non-porous support materials comprising a sample receiving zone for receiving a sample, and one or more detection zones comprising an immobilized specific binding reagent capable of forming a complex with a first target analyte on the target cell, if present in the sample. The method further comprises contacting the sample receiving zone with a sample containing or suspected of containing a target cell, flowing the sample to the detection zone, wherein the immobilized specific binding reagent forms a complex with the target cell, if present in the sample, and detecting the target cell. The target cell is detectable due to the presence of a label. The target cell can be labeled before the sample is applied to the sample receiving zone, before the target cell reaches a detection zone, and/or after the target cell reaches a detection zone. The detection of the label indicates the presence and/or amount of the target cell. In a further embodiment, the sample flows to a control zone comprising an immobilized control specific binding reagent, wherein the immobilized control specific binding reagent forms a detectable complex with one or more control cells. The detection of a control cell in the control zone indicates the validity of the test results.

In a further aspect, the method for detecting a target cell in a sample comprises providing a device comprising a support material comprising a detection zone comprising an immobilized specific binding reagent capable of forming a complex with a target analyte on the target cell, if present in the sample, contacting the detection zone with a sample containing or suspected of containing a target cell, wherein the immobilized specific binding reagent forms a complex with the target cell, and detecting the target cell. The target cell is detectable due to the presence of a label. The target cell can be labeled before and/or after the sample is applied to the detection zone. The detection of the label indicates the presence and/or amount of the target cell.

The present devices and methods can be used to detect an analyte on a target cell in any suitable sample.

The sample can be any biological sample containing intact cells, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). Typically, if biological tissues are used in the embodiments described herein, the cells comprising the tissues are at least partially disaggregated. Tissue disaggregation techniques are well known in the art.

In one embodiment of the invention, the sample is whole blood. The whole blood can be obtained from a finger-prick or other non-venous method, or can be obtained from a venous source. In one aspect, the whole blood is depleted of another cell type, such as, but not limited to, monocytes, using standard techniques. In certain embodiments, the whole blood sample is collected in a blood collection tube or blood collection container. In other embodiments, blood is applied directly to the device without storage in a blood collection tube or blood collection container. In a specific embodiment, blood is obtained by finger prick and applied directly to the sample receiving zone of the test device. This obviates the need for additional equipment (the blood collection tube or blood collection container) and increases the ease of use of the assay while decreasing the hands-on time necessary to perform the assay.

Traditional lateral flow assays often have a zone located upstream of the detection zone, which consists of a porous material capable of absorbing the sample and trapping cells, such that only the fluid portion of the sample enters the detection zone. These cell traps are intended to non-specifically trap all cells. In at least one embodiment of the present invention, the device does not comprise a zone upstream of the detection zone that absorbs and nonspecifically traps cells in a sample; rather, the device of the invention allows intact cells in the sample to flow into the detection zones. As stated above, these cell traps nonspecifically trap all cells. The cell depletion zones of the present invention are not cell traps as they are intended to trap a specific sub-population of cells, such as monocytes, while allowing other cell types to flow through and into the detection zone. In a specific embodiment of the present invention, the device does not comprise a material capable of nonspecifically trapping intact cells positioned upstream of the detection zone. Traditional cell traps can be made of fibrous materials such as paper, fleece, gel or tissue, cellulose, nitrocellulose, wool, glass fiber, asbestos, synthetic fibers, polymers, or mixtures thereof. Often, in traditional lateral flow assays, the cell traps are located in the sample receiving zone. However, cell traps may be located anywhere upstream of the detection zone such that only the liquid portion of a sample can enter the detection zone. Traditional cell traps trap cells by virtue of having pore sizes too small for cells to flow through, thus trapping the cells.

The present devices and methods can be used to detect any suitable analyte on a target cell.

Non-limiting examples of cells include animal cells, plant cells, fungi, bacteria, recombinant cells or cultured cells. Animal, plant cells, fungus, bacterium cells to be detected can be derived from any genus or subgenus of the Animalia, Plantae, fungus or bacterium kingdom. Cells derived from any genus or subgenus of ciliates, cellular slime molds, flagellates and microsporidia can also be detected. Cells derived from birds such as chickens, vertebrates such fish and mammals such as mice, rats, rabbits, cats, dogs, pigs, cows, ox, sheep, goats, horses, monkeys and other non-human primates, and humans can be detected by the present devices and methods.

For animal cells, cells derived from a particular tissue to organ can be detected. For example, connective, epithelium, muscle or nerve tissue cells can be detected. Similarly, cells derived from an accessory organ of the eye, annulospiral organ, auditory organ, Chievitz organ, circumventricular organ, Corti organ, critical organ, enamel organ, end organ, external female genital organ, external male genital organ, floating organ, flower-spray organ of Ruffini, Golgi tendon organ, gustatory organ, organ of hearing, internal female genital organ, internal male genital organ, intromittent organ, Jacobson organ, neurohemal organ, neurotendinous organ, olfactory organ, otolithic organ, ptotic organ, organ of Rosenmiiller, sense organ, organ of smell, spiral organ, subcommissural organ, subformical organ, supernumerary organ, tactile organ, target organ, organ of taste, organ of touch, urinary organ, vascular organs such as the vascular organ of lamina terminalis, vestibular organ, vestibulocochlear organ, vestigial organ, organ of vision, visual organ, vomeronasal organ, wandering organ, Weber organ and organ of Zuckerkandl can be detected. Preferably, cells derived from an internal animal organ such as brain, lung, liver, spleen, bone marrow, thymus, heart, lymph, blood, bone, cartilage, pancreas, kidney, gall bladder, stomach, intestine, testis, ovary, uterus, rectum, nervous system, gland, internal blood vessels, etc. can be detected.

Further, cells derived from any plants, fungi such as yeasts, bacteria such as eubacteria or archaebacteria can be detected. Recombinant cells derived from any eukaryotic or prokaryotic sources such as animal, plant, fungus or bacterium cells can also be detected. Cells from various types of body fluid such as blood, urine, saliva, bone marrow, sperm or other ascitic fluids, and sub-fractions thereof, e.g., serum or plasma, can also be detected. In one aspect, the target cell is a white blood cell. In another aspect, the target cell is a CD4+ lymphocyte. In another aspect, the target cell is a red blood cell such as a malaria infected red blood cell.

Any analyte associated with a target cell can be used to capture or detect the target cell. For example, the analyte can be any molecule on the surface of the target cell. Such molecules include proteins, peptides, hormones, steroids, or any other antigenic substance that is at least partially exposed on the surface of the cell. If the target cell is a CD4 lymphocyte, non-limiting examples of analytes include a CD4 antigen, a CD3 antigen, a CD8 antigen, a CD38 antigen, a CD14 antigen, and a CD45 antigen. It is contemplated that portions of the same antigen or different antigens can be used as analytes for the immobilized specific binding reagent and for the labeled specific binding reagent—a binding reagent coupled to a label. For example, a CD4 antigen can be used as a target analyte for the immobilized specific binding reagent, while a CD3 antigen can be used as the target analyte for the labeled specific binding reagent. In an alternative embodiment, a CD4 antigen can be used as a target analyte for the immobilized specific binding reagent, while the same or a different epitope of the CD4 antigen can be used as the target analyte for the detectable binding reagent.

If the target cell is a malaria infected red blood cell, the analyte is Plasmodium surface adhesive protein (called PfEMP1, for Plasmodium falciparum erythrocyte membrane protein 1). If the target cell is a B-cell producing immunoglobulin specific for a particular infective agent, the analyte is an B-cell receptor (BCR) molecule that specifically binds an antigen of the infective agent. If the target cell is a helper or cytotoxic T-cell specific for a particular antigen or peptide of interest, the analyte is a T-cell receptor (TCR) molecule that specifically binds a peptide of an infective agent/self antigen/cancer antigen which is presented in the context of an MHC multimer.

The descriptions herein utilize the term “specific binding reagent” with respect to a particular analyte; however, it is contemplated that a mixture of binding reagents specific to that analyte could be used. For example, if the CD4 antigen is the target analyte for the immobilized specific binding reagent, a mixture of antibodies specific to the CD4 antigen can be used. In such a case, the mixture of immobilized specific binding reagents directed to the same analyte is considered to be an immobilized specific binding reagent. Similarly, if the CD4 antigen is the target analyte for the labeled specific binding reagent, a mixture of antibodies specific to the CD4 antigen can be used, and such mixture of labeled specific binding reagents directed to the same analyte is considered to be a labeled specific binding reagent.

As described herein, a label is typically coupled to a binding reagent specific for an analyte on a target cell. When a sample containing target cells flows across the detection zone, it will be captured by binding to the immobilized specific binding reagent and become immobilized itself. When the binding reagent couples to a target cell captured in the detection zone, detection of the label indicates the presence and/or amount of target cells in the sample. The absence of a detectable label in the detection zone indicates the absence of target cells in the sample, or that the number of target cells present in the sample is below the detection limits of the assay. The labeled specific binding reagent can couple to a target cell either before and/or after the target cell forms a complex with the immobilized specific binding reagent in a detection zone of the devices of the invention. Labels include, but are not limited to, a radio-active material, a magnetic material, quantum dot, an enzyme, a liposome-based label, a chromophore, a fluorophore, a dye, a nanoparticle, a quantum dot or quantum well, a composite-organic inorganic nano-cluster, a colloidal metal particle, latex particles, or combinations thereof. In one aspect, the label comprises a colloidal gold particle, a colloidal silver particle conjugate, a latex particle, and/or a liposome-based particle. In certain aspects, the label is visible without the use of other instrumentation, other than the possible use of a magnifier. The magnifier can be an external magnifying lens or it can be incorporated into the device. For example, a magnifying lens could be incorporated into a housing that covers the device, such as in an optic opening around the detection zone and/or control zone. In another aspect, in embodiments where the detectable label emits light other than visible light, the magnifier or optical opening can also comprise a filter to detect emission of particular bands of ultraviolet or infra-red light. In certain embodiments, instruments can be used to detect the label. In a specific embodiment, an imaging device is used to quantitate the intensity of the label. For example, an imager such as the Kodak Imaging Station IS4000MM Pro can be used to quantitate the intensity of black latex particles.

The target cell can be labeled either before or after it reaches the detection zone. For example, the target cell can be contacted with the labeled specific binding reagent before it is applied to the device. Alternatively, the labeled specific binding reagent can be present at a labeling zone in the device, where it binds to the target cell when the sample comes into contact with the labeling zone. Generally, the labeled specific binding reagent will not be immobilized in the labeling zone, so that it does not capture the target cells in the labeling zone, but instead, moves as a complex with the target cells to the detection zone where the labeled target cell is then captured for visualization. In such embodiments, the labeled specific binding reagent can be air dried or lyophilized. In some embodiments, the labeled specific binding reagent is dried in the presence of a material that: a) stabilizes the labeled specific binding reagent; b) facilitates resuspension of the labeled specific binding reagent in the liquid; and/or c) facilitates mobility of the labeled specific binding reagent. Any suitable material can be used for stabilizing, or facilitating resuspension and/or the mobility of the labeled specific binding reagent. Exemplary materials include a protein, a peptide, a polysaccharide, a sugar, a polymer, a gelatin, and/or a detergent. In a further embodiment, the labeled specific binding reagent is added to the device after the target cells have been captured in the detection zone.

In one embodiment, the intensity of the label in a detection zone indicates the number of target cells in the sample. In another embodiment, detection of the label indicates the absence, presence and/or amount of the target cell. Knowing the approximate number or concentration of target cells in a sample can be useful information. For example, therapeutic intervention can only be warranted if a threshold concentration of a target cell is reached.

One aspect of the invention provides that the intensity of the label in a detection zone indicates the number and/or concentration of target cells in the sample. In another aspect, the amount of specific binding reagent applied to a detection zone is determined such that a zone can only specifically bind to a certain number of target cells. In one example, the intensity of the label correlates to a concentration of fewer than 200 CD4 cells/microliter of blood, to ˜200-350 CD4 cells/microliter of blood, to ˜350-500 CD4 cells/microliter of blood, or to greater than ˜500 CD4 cells/microliter of blood. In another example, a plurality of detection zones are present on the device, each containing a quantified amount of the specific binding reagent such an antibody to the CD4 antigen. The first detection zone will show a signal if there is fewer than ˜200-250 CD4 cells/microliter of blood depending on the sensitivity of the assay, while the second detection zone will show a signal if there are more than ˜350 CD4 cells/microliter of blood. Similarly, other detection zones which detect other amounts of the target cell can be generated, so that the devices are capable of semi-quantifying the amounts of target cell in the sample.

The amount of immobilized specific binding reagent can be increased, decreased, or can stay the same between the different adjacent zones. One of ordinary skill in the art can readily calculate the amount of specific binding reagent required to quantitate various levels of target cells in each detection zone.

In certain embodiments, whole blood is used as the sample. The whole blood can be treated to deplete it of certain unwanted components. For example, in the context of a test to detect CD4+ T-cells, it may be desirable to deplete the whole blood sample of monocytes which express the CD4 antigen. If the population of monocytes in the whole blood is increased due to infection, a test for CD4 lymphocytes can have a false positive due to the large numbers of CD4 positive monocytes. Monocyte depletion can be done either before the blood is applied to the devices of the invention, or after the blood has been applied. For example, a blood collection tube can be used to obtain the whole blood from a subject, where the blood collection tube contains a depletion specific binding reagent specific for monocytes. The blood that is placed in such a tube will be depleted of monocytes. In an alternative, the depletion specific binding reagent specific for monocytes can be attached to a magnetic bead. The magnetic beads are mixed with the whole blood to form a complex between the binding reagent and the monocytes, and the beads are separated from the blood using a magnet. In certain embodiments, the magnetic beads are “dry” (i.e. not suspended in a fluid) such that the whole blood sample is not diluted before application to the device. The content of the blood that is not bound to the magnet is monocyte depleted and is added to the device of the invention.

In another alternative, monocytes are depleted from the blood by a depletion zone on the device. Such depletion zones are typically upstream from a detection zone. A depletion zone will contain an immobilized depletion specific binding reagent, so that cells containing an antigen that binds to the depletion specific binding reagent will be immobilized on the device, and will not flow to the detection zone. For example, the depletion specific binding reagent specific for monocytes can be an antibody to CD14. In one aspect, the depletion zone can also be used as a control zone by detecting the monocytes that have been captured in the depletion zone. It is contemplated that greater than 20%, 30%, 40%, 50%, 60%, 70%, 80%, 90%, or 95% of the monocytes in the whole blood can be depleted from the whole blood. In certain embodiments, the sample receiving zone and the depletion zone are the same zone.

These types of methods and other similar methods can be used to deplete any specific component of the sample simply by varying the depletion specific binding reagent. For example, in the context of an assay to detect malaria infected red blood cells it may be desirable to deplete white blood cells. This can be done with depletion specific binding reagents to CD4, CD8, CD14 CD45 or any other cell surface analytes specific to white blood cells. In certain embodiments, the device of the invention comprises a cell depletion zone comprising an immobilized specific binding reagent that forms a complex with a specific sub-population of cells. As used herein, a population of cells includes all of the cells and cell types found in the sample as added to the device. A sub-population of cells includes any subdivision of a population with common, distinguishing characteristics. For example, white blood cells are a sub-population of a whole blood sample. Lymphocytes are also a sub-population of a whole blood sample. A sub-population of cells, such as white blood cells, can be further divided into more narrowly defined sub-populations, such as lymphocytes.

The invention further contemplates kits for detecting CD4 lymphocytes in a whole blood sample.

A kit for detecting target cells in a whole blood sample comprises labeled specific binding reagent specific for an analyte on the target cell, control cells, a diagnostic device comprising an immobilized specific binding reagent specific for an analyte on the target cell, and a diagnostic device comprising an immobilized control cell specific binding reagent. In one embodiment, the diagnostic device comprising an immobilized specific binding reagent, and the diagnostic device comprising an immobilized control cell specific binding reagent are the same device. In another embodiment, they are separate devices. In a further embodiment, the labeled target cell specific binding reagent is present on the device. In another embodiment, the kit for detecting target cells in a whole blood sample can further comprise a container for collecting the blood sample, a blood collection tube, and an anticoagulant.

In another aspect, a kit for detecting CD4 lymphocytes in a whole blood sample comprises labeled CD3 specific binding reagent and/or labeled CD4 specific binding reagent, control cells, a diagnostic device comprising an immobilized CD4 specific binding reagent, and a diagnostic device comprising an immobilized control cell specific binding reagent. In one embodiment, the diagnostic device comprising an immobilized CD4 specific binding reagent, and the diagnostic device comprising an immobilized control cell specific binding reagent are the same device. In another embodiment, they are separate devices. In a further embodiment, the labeled CD3 specific binding reagent and/or labeled CD4 specific binding reagent is present on the device. In another embodiment, the kit for detecting CD4 lymphocytes in a whole blood sample can further comprise a container for collecting the blood sample, a blood collection tube, and an anticoagulant.

In another aspect, the kit for detecting CD4 lymphocytes in a whole blood sample comprises a labeled CD3 specific binding reagent and/or a labeled CD4 specific binding reagent, control cells, a diagnostic device comprising a different immobilized CD4 specific binding reagent and an immobilized CD14 specific binding reagent, and a diagnostic device comprising an immobilized control cell specific binding reagent. In one embodiment, the diagnostic device comprising an immobilized CD4 specific binding reagent and an immobilized CD14 specific binding reagent, and the diagnostic device comprising an immobilized control cell specific binding reagent are the same device. In another embodiment, they are separate devices. The diagnostic device in such a kit can comprise one or more non-porous support materials capable of generating lateral flow of intact cells, the one or more non-porous support materials comprising a sample receiving zone for receiving a sample comprising a target cell, a first detection zone comprising the immobilized CD14 specific binding reagent, and a second detection zone comprising the different immobilized CD4 specific binding reagent. In another embodiment, the kit for detecting CD4 lymphocytes in a whole blood sample can further comprise a container for collecting the blood sample, a blood collection tube, and an anticoagulant.

In a further embodiment, the container for collecting blood can also comprise a depletion agent. In a specific embodiment, the depletion agent comprises magnetic beads coupled to a CD14 specific binding agent.

In a further embodiment, the labeled CD3 specific binding reagent and/or labeled CD4 specific binding reagent is located on or in the device in a non-covalent manner. As an example, the labeled CD3 specific binding reagent and/or labeled CD4 specific binding reagent can be applied to an absorbent material which is attached to the chip, such that the labeled CD3 specific binding reagent and/or labeled CD4 specific binding reagent is not itself covalently attached to the chip. Typically, the sample receiving zone, the first detection zone, and the second detection zone are arranged in the one or more support materials such that the sample is capable of lateral flow sequentially across the sample zone, across the first detection zone, and then to the second detection zone.

In a third aspect, the kit for detecting CD4 lymphocytes in a whole blood sample comprises a container for collecting blood, a blood collection tube comprising means for depleting monocytes from a blood sample, an anticoagulant, labeled CD3 specific binding reagent and/or labeled CD4 specific binding reagent, control cells, a diagnostic device comprising a different immobilized CD4 specific binding reagent, and a diagnostic device comprising an immobilized control cell specific binding reagent. In one embodiment, the diagnostic device comprising an immobilized CD4 specific binding reagent, and the diagnostic device comprising an immobilized control cell specific binding reagent are the same device. In another embodiment, they are separate devices. In a further embodiment, the labeled CD4 specific binding reagent is present on the device.

Typically, the anticoagulant is present in the container for collecting blood. The blood collection tube can be integral to the container for collecting blood, or it can be separate from the container. The detection of a control cell in the control zone indicates the validity of the test results. The kit can further comprise a washing container and a washing solution. The kit can comprise a red blood cell lytic reagent, such as ammonium chloride or lithium salts such as lithium thiocyanate. The blood cell lytic reagent can be present in the interior of the blood collection tube, in the container for collecting blood, or in the detection device itself.

Both sample liquid and/or other liquid can be used to transport the target cells and/or the labeled specific binding reagent to the detection zone, to the control zone, to the distal end of the device, or to an absorbent pad downstream that is in fluid communication with the device. In one example, a sample liquid alone is used to transport the target cell and/or the labeled specific binding reagent to the desired location. This is often used when sufficient sample volume is available. In another example, a developing liquid is used to transport the target cell and/or the labeled specific binding reagent to the desired location. A developing liquid is often used to transport an analyte in a non-liquid, e.g., solid, sample. For example, a tissue can be disaggregated and suspending in a developing liquid. A developing liquid also can be used when a liquid sample, blood sample, is tested but the sample volume itself is not sufficient to transport the target cell and other reagents or substances to the desired location.

In a separate aspect, the kit for detecting CD4 lymphocytes in a whole blood sample comprises a diagnostic device comprising an immobilized monocyte specific binding reagent in a monocyte depletion zone near the sample addition zone, immobilized CD4 specific binding reagent in the detection zone, and a diagnostic device comprising an immobilized control cell specific binding reagent. In one embodiment, the diagnostic device comprising an immobilized CD4 specific binding reagent, and the diagnostic device comprising an immobilized control cell specific binding reagent are the same device. In another embodiment, they are separate devices. In a further embodiment, the labeled CD4 T cell specific binding reagent is present on the device. In a further embodiment, the control cells are present on the device. In certain embodiments, there is no need for a separate blood collection tube; rather a specific volume of blood is added directly to the sample addition zone.

### C. Methods to Immobilize a Specific Binding Reagent

It is contemplated that the specific binding reagent is immobilized on the support material. A binding reagent can be immobilized on the support material by any suitable methods. For example, binding reagent can be immobilized by absorption, adsorption, by non-covalent methods such as hydrophobic interactions, as well as by electrostatic attraction, by covalent binding to the support material, or by attaching the binding reagent to another substance or particle that is immobilized to the desired location on the support material. In one aspect of the invention, the binding reagent is covalently attached directly or indirectly to the support material.

Covalent attachment can be accomplished using any known technique. For example, the surface of a support material can be chemically modified to permit covalent conjugation to a specific binding reagent. Alternatively, the surface of a support material can be coated with a substance that permits covalent conjugation to a specific binding reagent. It is generally preferable to keep the surface of the support material hydrophilic to prevent the hydrophobic adsorption of non-targeted proteins onto the surface, which could impede visualization of the target cells.

In one example, a polyacrylonitrile membrane is chemically modified. Before modification, it contains nitrile groups (—CN) and is hydrophobic. The nitrile groups are hydrolyzed to form amido groups (—CONH₂). A functional group is then introduced on the surface of the membrane by converting the obtained amido groups (—CONH₂) to hydrazo groups (—NH—NH₂). A Schiff base is formed between the membrane surface hydrazo groups and glutaraldehyde. The presence of aldehyde groups on the surface of the support material permits the covalent conjugation of specific binding reagents.

Other membranes can similarly be modified. For example, Whatman S14 and S17 and Porex® X4588 can be modified through the formation of polymeric glutaraldehyde within the body of the material. Again, the presence of aldehyde groups on the support material permits the covalent conjugation of specific binding reagents.

Other chemically reactive groups can be present on the surface of the support materials that covalently bind specific binding reagents. For example, various organic and inorganic groups can be used to form chemically reactive groups, such as hydroxyl, carboxyl, amino, sulphonyl, thiol, and aldehyde groups. Table 1 shows non-limiting examples of chemically reactive groups and corresponding target functional groups. One of ordinary skill in the art would readily be able to modify the support materials to include one or more of these or similar chemically reactive groups.

TABLE 1

| Reactive Group | Target Functional Group |
|---|---|
| Aryl Azide | Nonselective (or primary amine) |
| Carbodiimide | Amine/carboxyl |
| Hydrazide | Carbohydrate (oxidized) |
| Hydroxymethyl Phosphine | Amine |
| Imidoester | Amine |
| Isocyanate | Hydroxyl (non-aqueous) |
| Carbonyl | Hydrazine |
| Maleimide | Sulfhydryl |
| NHS-ester | Amine |
| PFP-ester | Amine |
| Psoralen | Thymine (photoreactive |
| | intercalator) |
| Pyridyl Disulfide | Sulfhydryl |
| Vinyl Sulfone | Sulfhydryl, amine, hydroxyl |
| Carbonyl | Hydrazine |

Non-porous materials can be treated with substances that permit covalent conjugation to specific binding reagents. In one example, the devices comprising one or more non-porous support materials are coated with oxidized dextran, prepared as described previously (See U.S. Pat. No. 5,466,609). In other examples, the devices comprising one or more non-porous support materials can be coated with silicon, a metal or other substances. The support material can be given a hydrophilic coating, for example, by subjecting it to an oxidative treatment, such as by gas plasma treatment, coating with a hydrophilic substance such as silicon oxide, hydrophilic polymers such as dextran, polyethylene glycol, heparin and derivatives thereof, detergents, biologic substances such as polymers, etc.

### D. Exemplary Embodiments

This disclosure discusses an improved lateral flow immunoassay that is designed for low cost production, high sensitivity, reliability, broad dynamic range, and compatibility with low-cost, hand-held fluorescence readers.

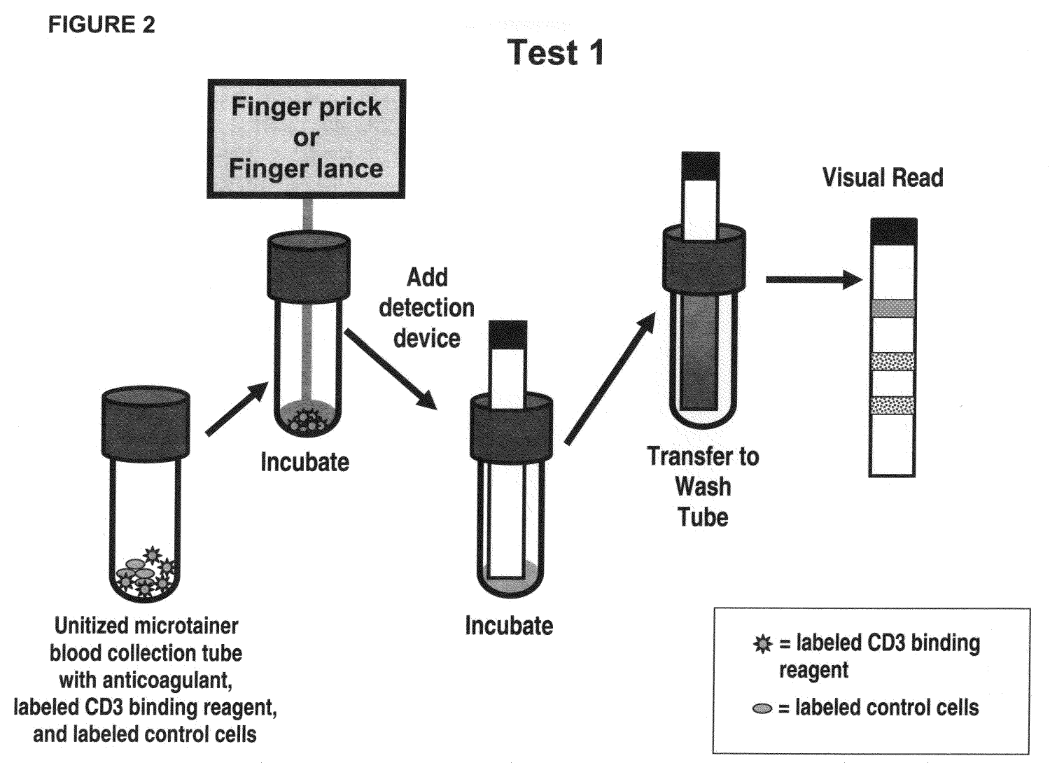

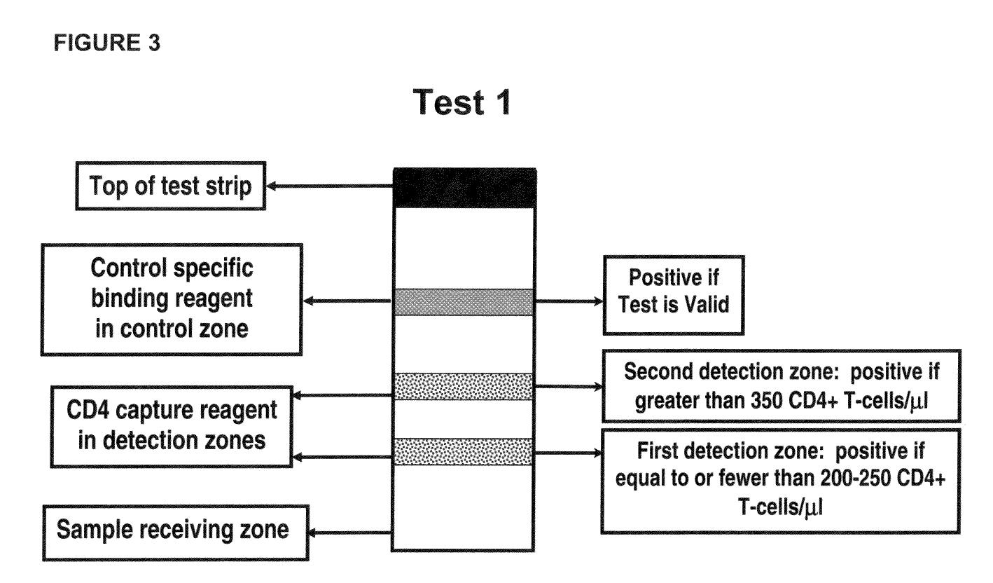

FIGS. 2 and 3 show one exemplary embodiment of a device and methods of the present invention.

A blood collection container is provided. The blood collection container can be a unitized container comprising a blood collection tube, such as the Multivette® 600 (Sarstedt) or the Microvette® 100/200 (Sarstedt). Alternatively, the blood collection tube can be separate from the blood collection container. In this embodiment, blood is collected from a finger prick or finger lance.

For reproducibility, a consistent amount of a sample is applied to the test device. In one aspect, a blood collection tube is used to control the amount of blood collected. In various aspects, a blood collection tube is selected to collect ˜50 μl of blood, ˜100 μl of blood, ˜150 μl of blood, ˜200 μl of blood, or any other selected amount. Alternatively, any volume of blood (or other sample) can be collected, and a pipette or other tool used to transfer the requisite amount of blood (or other sample) to the test device. In certain embodiments, sample blood from a finger prick is applied directly to the device without collecting or storing the sample in a blood collection tube or blood collection container.

In this embodiment, a controlled volume of blood enters the blood collection container, which previously contained an anticoagulant, a labeled specific binding reagent, and labeled control cells. The labeled specific binding reagent is specific for an analyte on the target cells. In this embodiment, the target cell is a CD4 lymphocyte, and the labeled specific binding reagent is a labeled CD3 binding reagent. The control cells provide an indication of a valid test result. Sample control cells include, for example, Cyto-trol™ cells (Beckman Coulter) and Immuno-trol™ cells (Beckman Coulter). Alternatively, the control cells are a component of the sample blood itself, such as CD45+ lymphocytes, CD8+ lymphocytes, or granulocytes. These components are permitted to incubate for a period of time, sufficient to permit the labeled specific binding reagent to form a complex with the target cells suspected of being present in the sample blood from the subject.

The detection device is then contacted with the sample, such as by introducing a portion or all of the device into the blood collection container. FIG. 2 displays the use of a lateral flow device. The sample receiving zone of the device is contacted with the sample. A plurality of sample receiving zones can be provided, but for the sake of simplicity, the pictured embodiment only shows one sample receiving zone. The sample flows from the sample receiving zone downstream to various other zones in the device, including at least one detection zone, and optionally, at least one control zone, via lateral flow. The device is then visually read to determine the presence, absence and/or amount of the label, which provides an indication of the presence, absence and/or amount of target cell contained in the sample. Non-limiting labels include colloidal gold particles, colloidal silver particles, latex beads, and liposome-based labels, any of which can be conjugated to a reagent specific for an analyte on the target cells or control cells. Optionally, the device is washed prior to visualization. Any washing buffer can be used so long as it does not interfere with the visualization of the label, and does not affect the stability or formation of the complex between the label, the target cell and the target analyte specific binding reagent. Typically, the detection is by visual means, and does not require the use of additional instrumentation, other than the possible use of a magnifier or corrective lenses.

An exemplary device is shown in more detail in FIG. 3. As illustrated, the device comprises a support material in the shape of a strip, however, this is a non-limiting form, and the device can be shaped in any manner so long as it achieves its purpose. The sample is applied at the bottom of the device (test strip) and the fluid containing the target cells flows to the top of the test strip via lateral flow.

This particular device contains two detection zones and one control zone, however, additional detection and control zones are also contemplated. Devices containing one, two, three, four or more detection zones are contemplated. Moreover, the control zone can be present on a completely separate device, and need not be incorporated into the device that detects the presence, absence, and/or amount of the target cells.

The control zone contains an immobilized control specific binding reagent that captures the labeled control cells. The control zone is positive if the device works properly, indicating a valid test result.

In this particular embodiment, the first detection zone is the zone closest to the sample receiving zone. The second detection zone is downstream from the first detection zone. Both detection zones contain immobilized target cell specific binding reagents in differing amounts. In this scenario, the target cell specific binding reagent is CD4 target cell specific binding reagent such as an anti-CD4 antibody.

The sample is transported via lateral flow from the sample receiving zone, across the detection zones, and across the control zones. Lateral flow devices typically contain an end region beyond the detection and/or control zone where unbound cells and reagents will normally migrate due to capillary action of the liquid if they do not bind to the immobilized specific bind reagents.

The label is then detected.

The first detection zone comprises a quantity of immobilized CD4 specific binding reagent sufficient to capture enough CD4+ cells that the cells can be detected when labeled with a CD4 cell specific labeled binding reagent when equal to or fewer than 200-250 CD4+ cells/μl are present in the sample. The second detection zone comprises a quantity of immobilized CD4 specific binding reagent sufficient to capture enough CD4+ cells that the cells can be detected when labeled with a CD4 cell specific labeled binding reagent only when greater than or equal to about 350 cells/μl are present in the sample. If a label is detected only in the first detection zone, the sample contains equal to or fewer than 200-250 CD4 cells/microliter of blood. If a label is detected in both the first and second detection zones, the sample contains greater than 350 CD4 cells/microliter of blood. If no label is detected, the sample contains either no CD4 cells or the number of CD4 cells/microliter of blood is below the detection limit of the test. A decision about whether to administer a therapeutic can be made based upon whether the sample contains a threshold number or concentration of cells, as described in more detail below.

In this embodiment, the labeling of CD3+ cells in the blood sample and capture by CD4 specific binding reagents on the device theoretically eliminates the need to deplete the blood sample of monocytes or the need to purify the CD4 T cells.

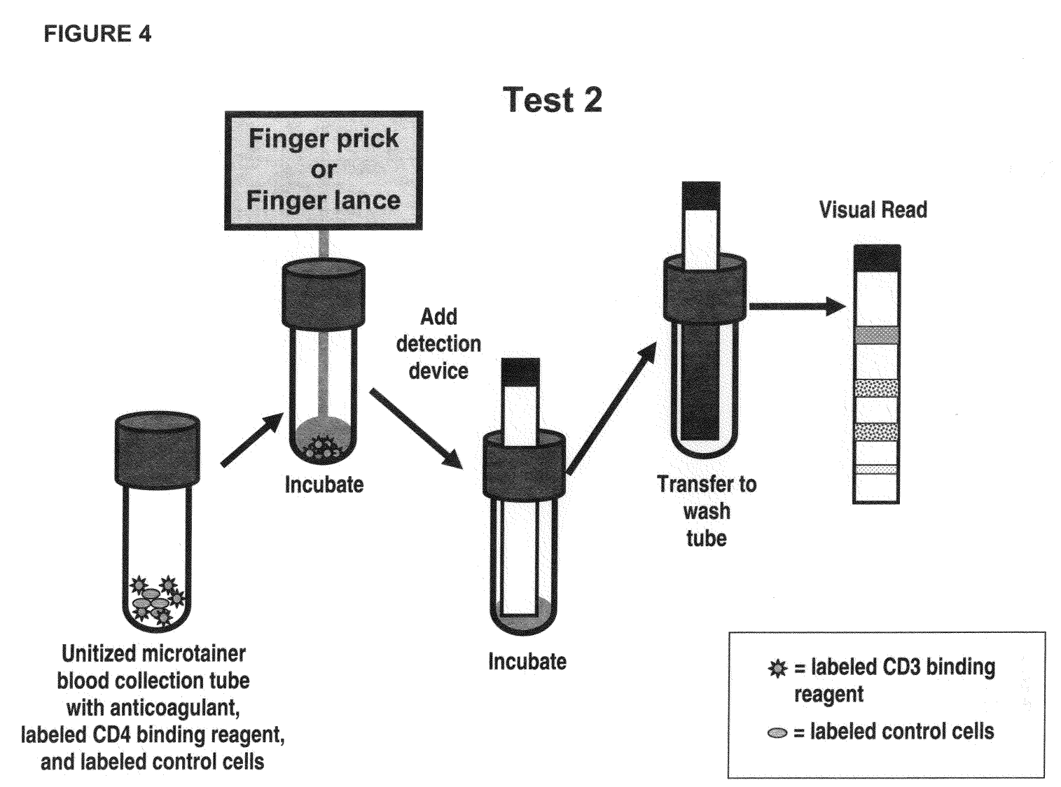

The embodiments illustrated in FIGS. 4-9 contain many of the features described in FIGS. 2 and 3, and thus the description above and below applies equally to all of the devices and methods described herein.

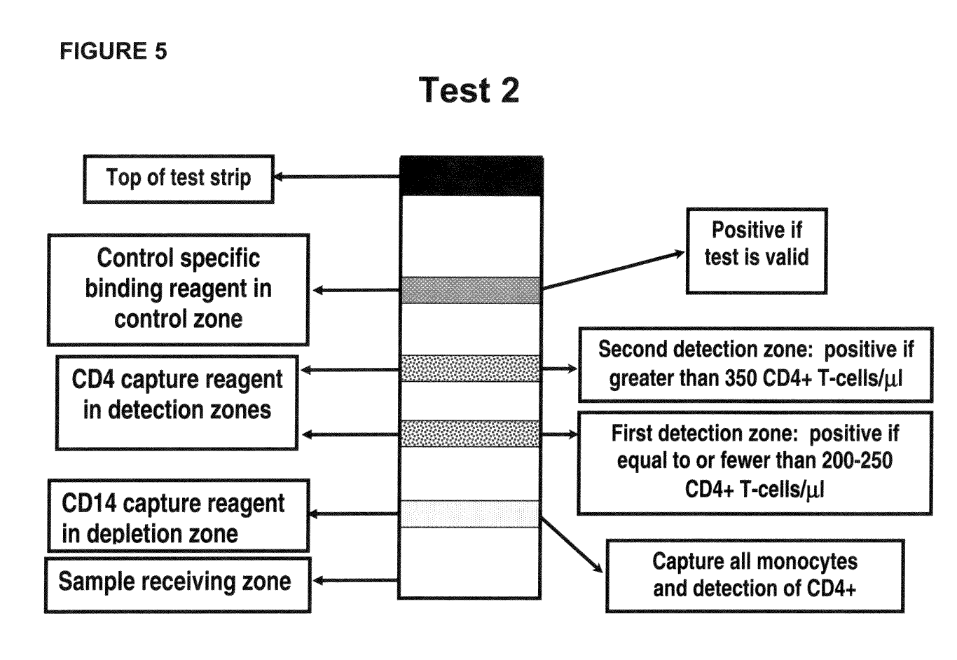

FIGS. 4 and 5 show another exemplary embodiment of a device and methods of the present invention. It differs from FIGS. 2 and 3 in that a different labeled specific binding reagent is used, and the whole blood is monocyte depleted through the use of an immobilized depletion specific binding reagent in a depletion zone on the device.

A blood collection container and blood collection tube are provided as in FIG. 2. Blood is collected from a finger prick or finger lance. It enters the blood collection container, which previously contained an anticoagulant, a labeled specific binding reagent, and labeled control cells. The labeled specific binding reagent is specific for an analyte on the target cells. In the embodiment shown, the target cell is a CD4 lymphocyte, and the labeled specific binding reagent is a labeled CD4 binding reagent. The control cells provide an indication of a valid test result. These components are permitted to incubate for a period of time, sufficient to permit the labeled specific binding reagent to form a complex with the target cells suspected of being present in the whole blood sample.

The detection device is then added to the blood collection container. FIG. 4 displays the use of a lateral flow device in the shape of a strip. The sample receiving zone of the device is contacted with the sample. The sample flows from the sample receiving zone downstream to various other zones in the device via lateral flow. The device is then visually read to determine the presence, absence and/or amount of the label, which provides an indication of the presence, absence and/or amount of target cell contained in the sample. Optionally, the device is washed prior to visualization. Again, the detection is typically by visual means.

The device is shown in more detail in FIG. 5. As illustrated, the device comprises a support material in the shape of a strip, however, this is a non-limiting form, and the device can be shaped in any manner so long as it achieves its purpose. The sample is applied at the bottom of the device (test strip) and the fluid containing the target cells flows to the top of the test strip.

This particular device contains two detection zones, a depletion zone and one control zone, however, additional detection, depletion and control zones are also contemplated. The sample is transported via lateral flow from the sample receiving zone, across the depletion zone, the detection zones, and across the control zone. The label is then detected.

The control zone contains an immobilized control specific binding reagent that captures the labeled control cells. The control zone is positive if the device works properly, indicating a valid test result.

The depletion zone contains an immobilized depletion specific binding reagent that captures cells that form a complex with the binding reagent. In this particular example, the immobilized depletion specific binding reagent is a CD14 specific binding reagent. For example, approximately 0.01-500 μg of a CD14 specific antibody is coupled to the solid support in the depletion zone. The amount of specific binding agent required to deplete a sample of a type is cell can be readily determined by one of ordinary skill in the art. The CD14 specific binding reagent captures and immobilizes monocytes, thus depleting monocytes from the sample that is flowing across the device and decreasing the possibility of a false positive result in the detection zone(s).

The first detection zone is the zone closest to the sample receiving zone. The second detection zone is downstream from the first detection zone. Both detection zones contain immobilized CD4 specific binding reagent in differing amounts. In this scenario, if a label is detected in the first detection zone, the sample contains equal to or fewer than 200-250 CD4 cells/microliter of blood. If a label is detected in both the first and second detection zones, the sample contains greater than 300-350 CD4 cells/microliter of blood. If no label is detected, the sample contains either no CD4 cells or the number of CD4 cells/microliter of blood is below the detection limit of the test.

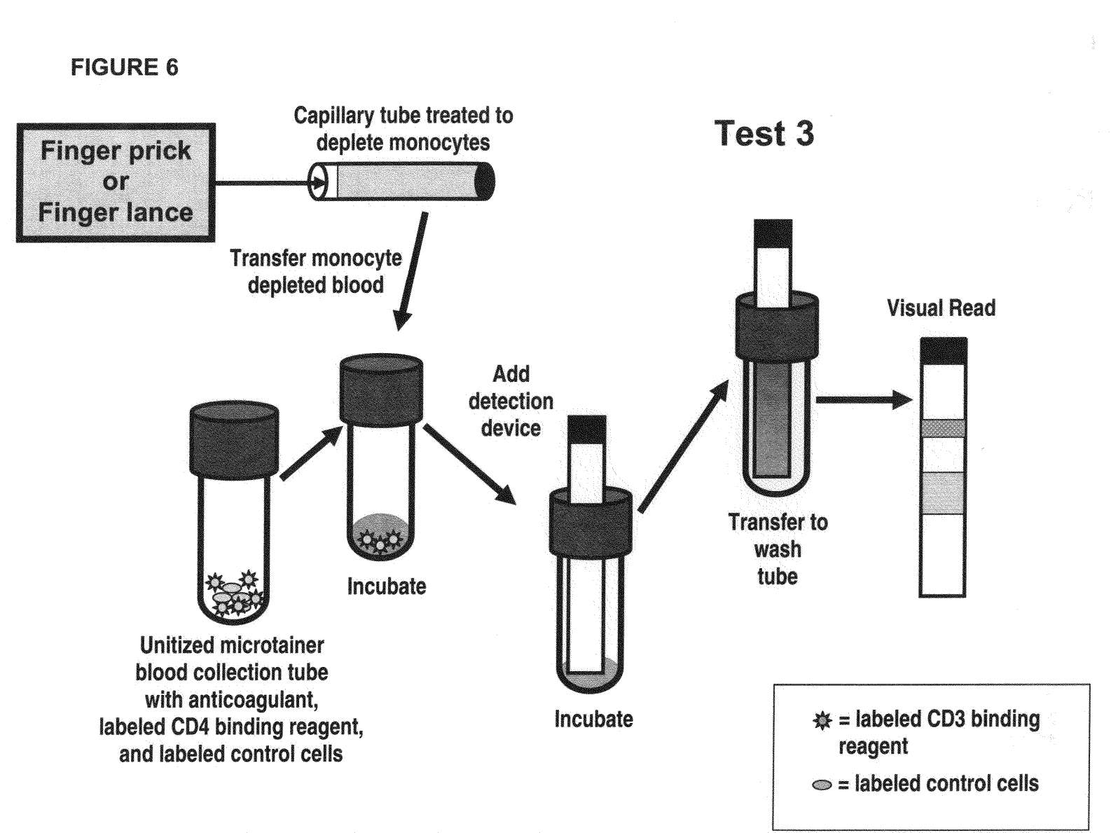

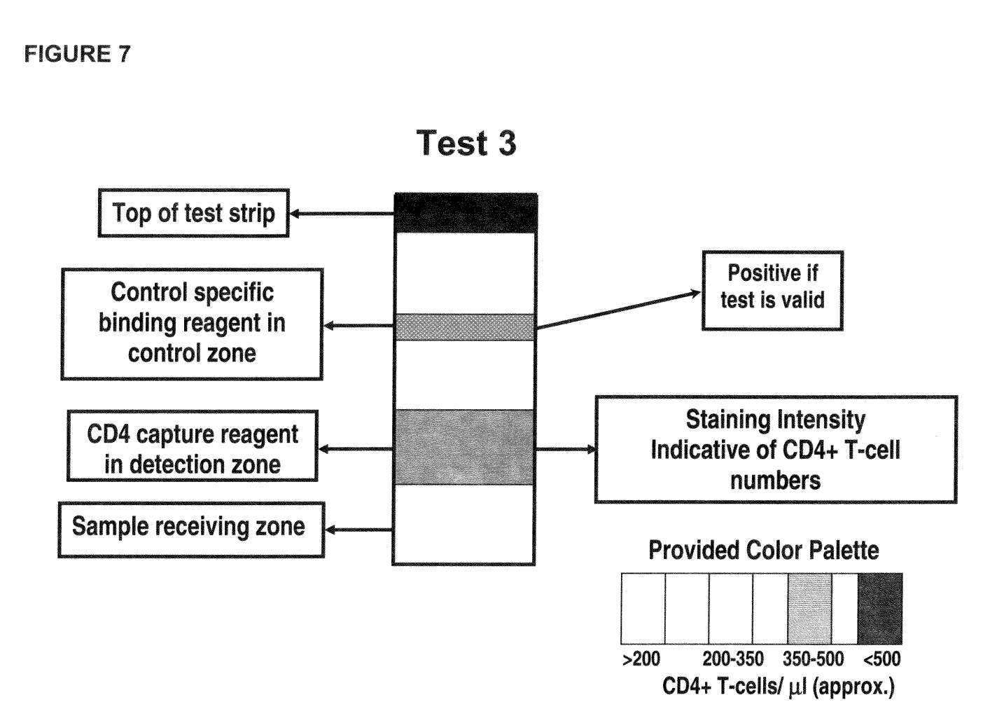

FIGS. 6 and 7 show a third exemplary embodiment of a device and methods of the present invention.

A blood collection container and blood collection tube are provided as in FIGS. 2 and 4. Blood is collected from a finger prick or finger lance using a blood collection tube. The collection tube (capillary tube) contains a depletion specific binding reagent such as a CD 14 specific binding reagent to deplete monocytes from the whole blood. The monocyte depleted blood enters the blood collection container, which previously contained an anticoagulant, a labeled specific binding reagent, and labeled control cells. The labeled specific binding reagent is specific for an analyte on the target cells. In this embodiment, the target cell is a CD4 lymphocyte, and the labeled specific binding reagent is a labeled CD4 binding reagent. The control cells provide an indication of a valid test result. These components are permitted to incubate for a period of time, sufficient to permit the labeled specific binding reagent to form a complex with the target cells suspected of being present in the whole blood sample.

The detection device is then added to the blood collection container. FIG. 6 displays the use of a lateral flow device in the shape of a strip. The sample receiving zone of the device is contacted with the sample. The sample flows from the sample receiving zone downstream to various other zones in the device via lateral flow. The device is then visually read to determine the presence, absence and/or amount of the label, which provides an indication of the presence, absence and/or amount of target cell contained in the sample. Optionally, the device is washed prior to visualization. Again, the detection is typically by visual means.

The device is shown in more detail in FIG. 7. As illustrated, the device comprises a support material in the shape of a strip, however, this is a non-limiting form, and the device can be shaped in any manner so long as it achieves its purpose. The sample is applied at the bottom of the device (test strip) and the fluid containing the target cells flows to the top of the test strip.

This particular device contains one detection zone, and one control zone, however, additional detection and control zones are also contemplated.

The control zone contains an immobilized control specific binding reagent that captures the labeled control cells. The control zone is positive if the device works properly, indicating a valid test result.

As described, this embodiment shows only one detection zone, although the use of multiple detection zones is contemplated. The intensity of the label detected in the detection zone provides an indication of the presence, absence, and/or amount of the target cell in the sample. For example, a color palette can be provided in the kit, correlating the strength of the visualized signal with the amount or concentration of target cells in the sample. In this embodiment, various intensities can be used to indicate the presence of fewer than ˜200 CD4 T cells/microliter of blood, ˜200-350 CD4 T cells/microliter of blood, ˜350-500 T CD4 cells/microliter of blood, or greater than ˜500 CD4 T cells/microliter of blood. Other cut-offs are also contemplated for example, 700 CD4 T cells/microliter of blood for initiation of therapy in pediatric populations This type of detection zone can also be employed in the embodiments described in FIGS. 2-5.

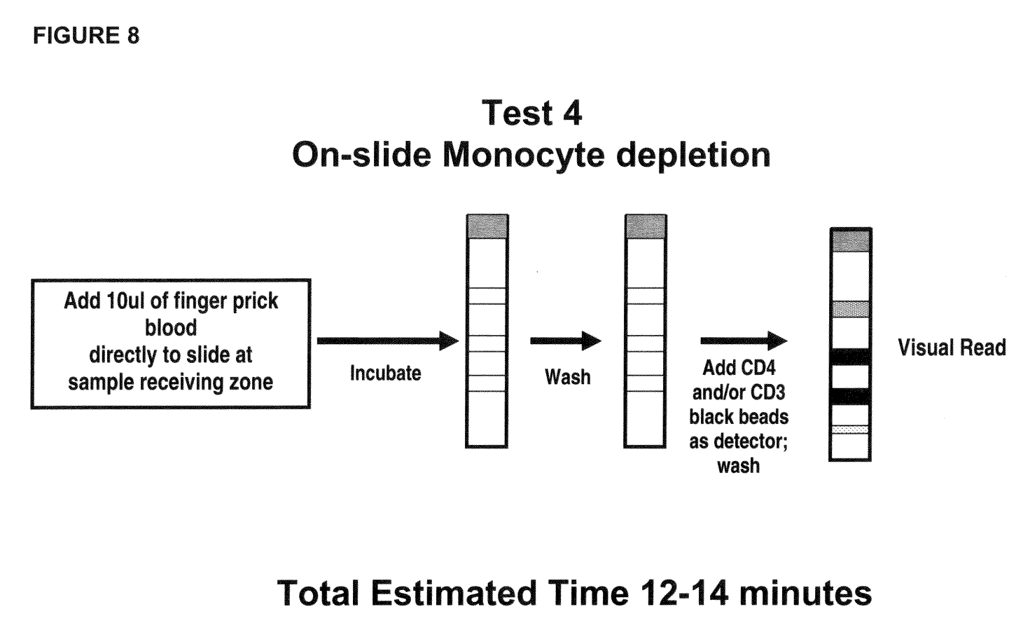

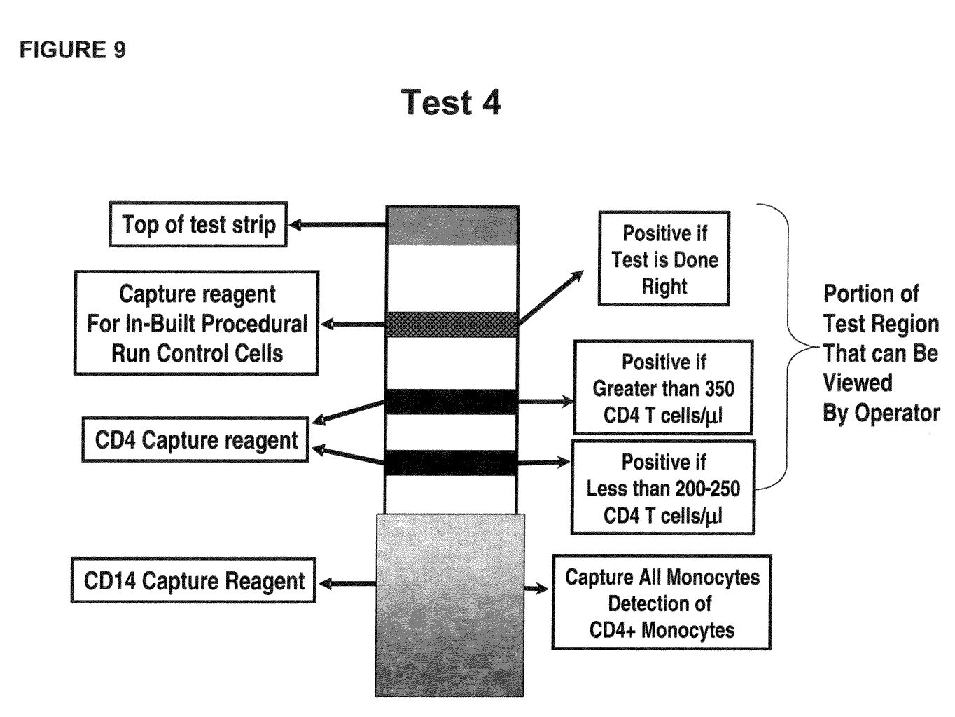

FIGS. 8 and 9 show a fourth exemplary embodiment of a device and methods of the present invention.

Blood is collected from a finger prick or finger lance and directly applied to the assay device. FIG. 9 displays the use of a lateral flow device in the shape of a strip. The sample receiving zone of the device is contacted with the sample of whole blood applied directly to the device. The sample flows from the sample receiving zone downstream to various other zones in the device via lateral flow. The device is then visually read to determine the presence, absence and/or amount of the label, which provides an indication of the presence, absence and/or amount of target cell contained in the sample. Optionally, the device is washed prior to visualization. The detection is typically by visual means.

This particular device contains one monocyte depletion zone, two detection zones, and one control zone, however, additional detection and control zones are also contemplated.

The first detection zone is the zone second closest to the sample receiving zone. The second detection zone is downstream from the first detection zone. Both detection zones contain immobilized specific binding reagent in differing amounts, such as an immobilized CD4 specific binding reagent. In this scenario, if a label is detected in the first detection zone, the sample contains equal to or fewer than 200-250 CD4 cells/microliter of blood. If a label is detected in both the first and second detection zones, the sample contains greater than 300-350 CD4 cells/microliter of blood. If no label is detected, the sample contains either no CD4 cells or the number of CD4 cells/microliter of blood is below the detection limit of the test.

The control zone contains an immobilized control specific binding reagent that captures the labeled control cells. The control zone is positive if the device works properly, indicating a valid test result.

The monocyte depletion zone is the zone closest to the sample receiving zone. It contains an immobilized monocyte specific binding reagent to capture monocytes in the sample. The monocyte specific binding reagent can be a reagent that forms a complex with CD14.

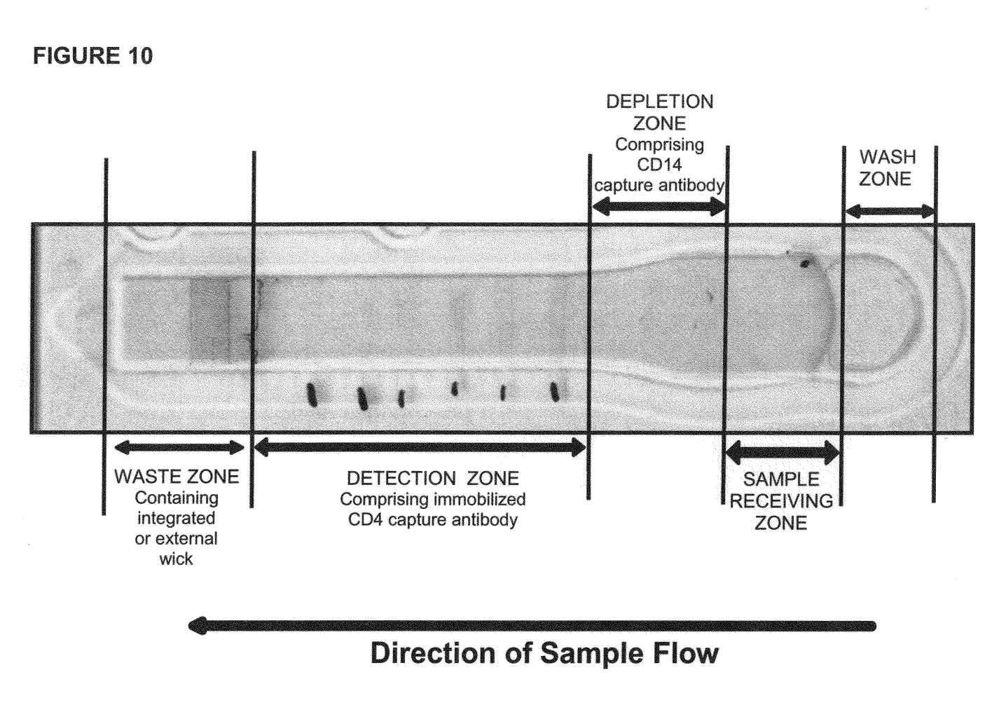

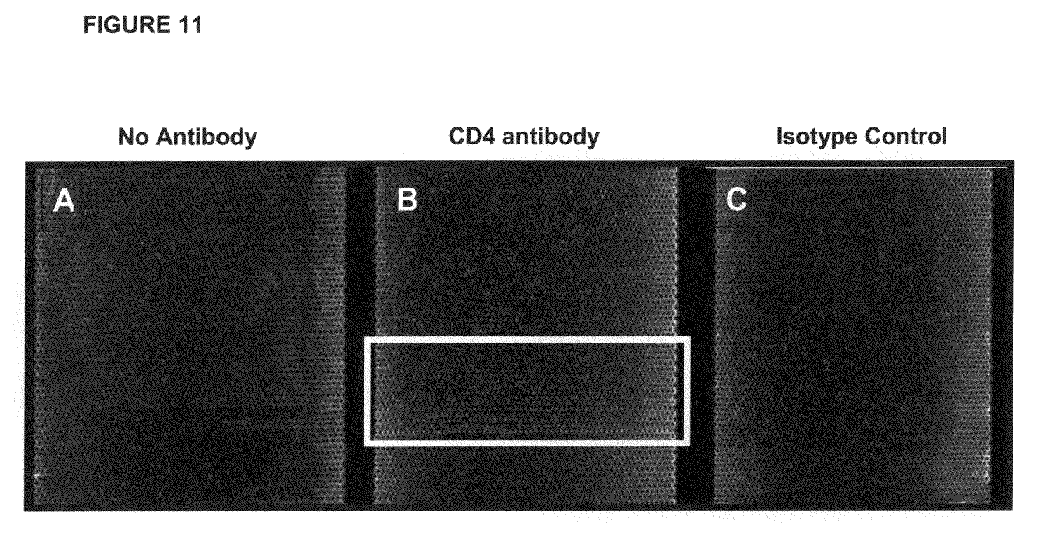

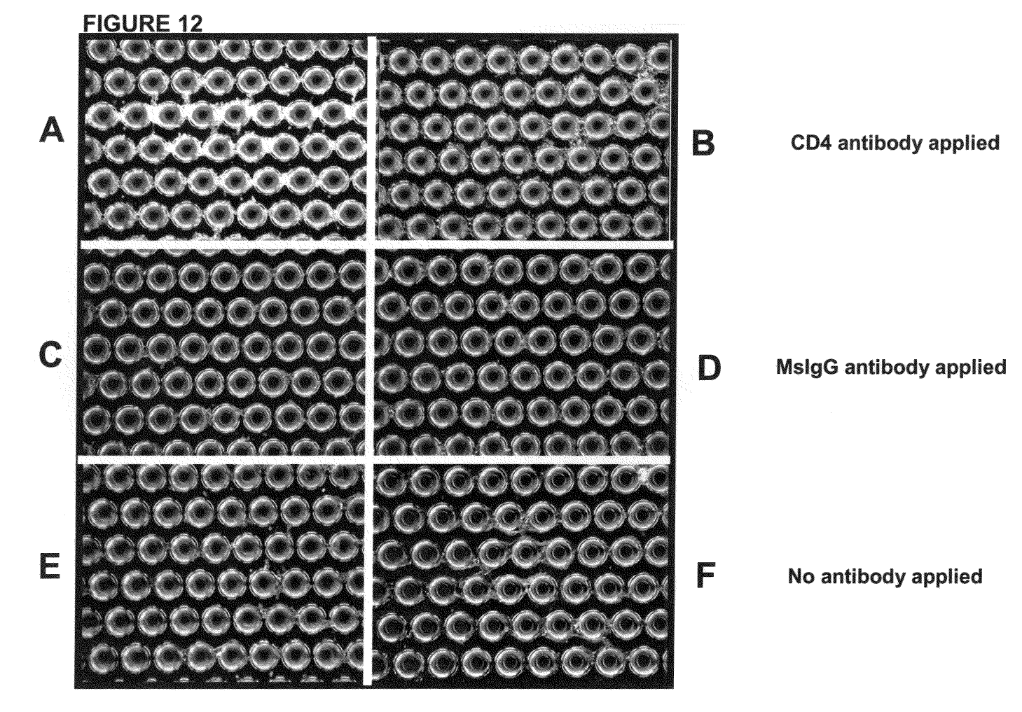

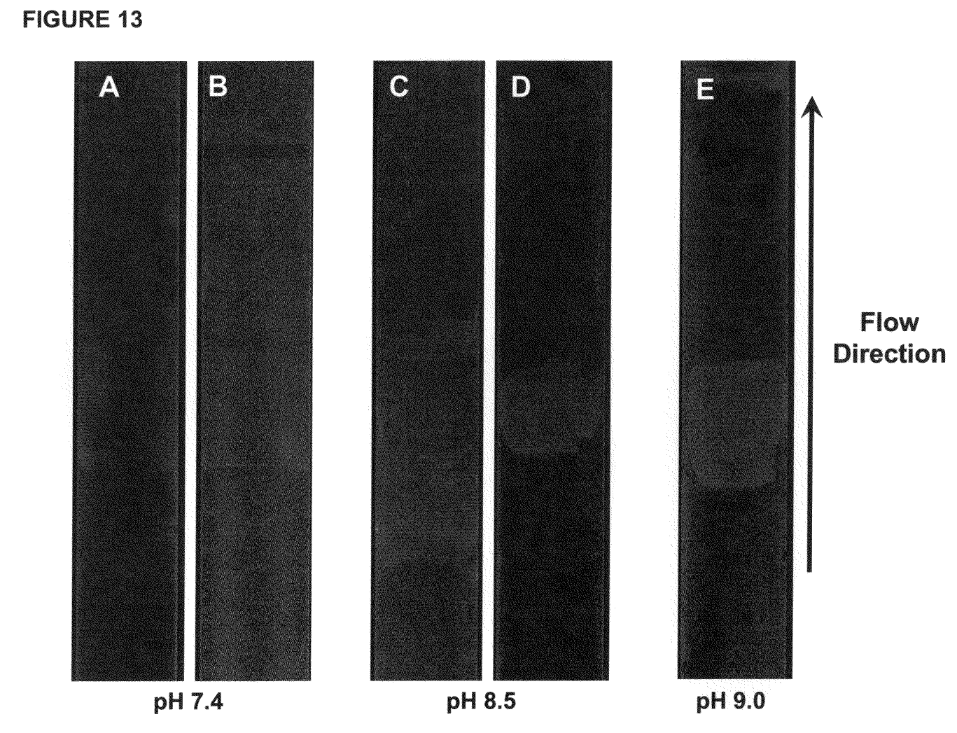

One exemplary embodiment of a non-porous support material used in the methods of the invention is the chip shown at FIG. 10. This is a commercial chip purchased from Amic AB. It is a plastic chip containing a highly ordered array of micropillars that drive flow of liquid in an open channel by capillary action. The micropillars are substantially vertical protrusions from the surface of the substrate. The devices are structured to permit lateral flow of whole blood through the micropillars without structurally impeding the flow of leukocytes. The flow of liquid across the chip is controlled by the pillar geometry and the channel length. Preferably, the device permits flow of between about 10 to about 200 μl of whole blood. In one embodiment, the micropillars are spaced about 30 μm apart, measuring from the edges of adjacent micropillars.

FIG. 10 shows an exemplary device comprising a wash zone, a sample receiving zone, a cell depletion zone, a detection zone (also called the test zone) and a waste zone. The detection zone comprises six distinct stripes of antibody to a CD4 epitope. The detection zone need not be placed in this particular location, but can be located at any point downstream of the sample receiving zone. In this embodiment, the micropillars are all equally spaced, however, the invention also contemplates the use of devices where the micropillars are not equally spaced. For example, the spacing of the micropillars can vary between the different zones, or within a zone. In this exemplary device, sample is applied to the sample receiving zone, and flows via lateral flow across the cell depletion zone, across the detection zone and then to the waste zone. Optionally, developing liquid is applied to the chip. For example, a developing liquid can be applied to the sample zone, to the wash zone, and/or to various positions in the detection zone. In one aspect, aliquots of a developing liquid agent are applied to the sample zone, one in the front of detection zone and one at the end of the detection zone. Any suitable developing liquid can be used, such as a buffer containing a detergent.

A wick can be integral with the device, or can be added to the device. For example, a cellulose wick can be added to the end of the waste zone of the device shown in FIG. 10, and wet with water or any other developing liquid to thereby act as a wick. Wicks are often made from non-woven, cellulose fiber sheets. These pads can be manufactured in a variety of thicknesses and densities to suit the needs of the assay. The wick pulls fluid off of the chip or membrane to allow the capillary flow to continue in the proper direction and at the proper rate. The use of a wick can prevent the sample and buffer from flowing back down the chip or membrane, which could raise the background or possibly cause false positives. This can also occur if the wick selected for the volumes sample involved is inadequate. Other types of wicks are also contemplated by the invention.

Although the embodiments described above are directed to a sandwich type assay, one of ordinary skill in the art could readily adapt the devices and methods to perform competition and/or inhibition type assays.

The devices and methods described herein are useful for determining and monitoring the presence, absence and/or amount of a target cell in a sample. In various embodiments, they are useful for ascertaining the CD4 lymphocyte count, the CD4 percentage, and/or the CD4:CD8 ratio in subjects infected with HIV. The CD4 cell (CD4+ T helper lymphocyte and CD4+ monocyte) is the primary target for HIV infection because of the affinity of the virus for the CD4 surface marker. The CD4 lymphocyte coordinates a number of important immunologic functions, and a loss of these functions results in progressive impairment of the immune response. Studies of the natural history of HIV infection have documented a wide spectrum of disease manifestations, ranging from asymptomatic infection to life-threatening conditions characterized by severe immunodeficiency, serious opportunistic infections, and cancers. Other studies have shown a strong association between the development of life-threatening opportunistic illnesses and the absolute number (per microliter of blood) or percentage of CD4 lymphocytes. As the number of CD4 lymphocytes decreases, the risk and severity of opportunistic illnesses increase.

There are four clinical stages of HIV Infection:
Stage I: HIV disease is asymptomatic and not categorized as AIDS.
Stage II: include minor mucocutaneous manifestations and recurrent upper respiratory tract infections.
Stage III: includes unexplained chronic diarrhea for longer than a month, severe bacterial infections and pulmonary tuberculosis.
Stage IV: includes toxoplasmosis of the brain, candidiasis of the esophagus, trachea, bronchi or lungs and Kaposi's sarcoma; these diseases are used as indicators of AIDS.

Measures of CD4 lymphocytes are thus used to guide clinical and therapeutic management of HIV-infected persons. According to public health guidelines, preventive therapy should be started when an HIV-positive person who has no symptoms registers a CD4 count under 200 cells/μl. Some physicians will opt to consider treatment earlier, at 350 cells/μl. The Centers for Disease Control and Prevention considers HIV-infected persons who have CD4 counts below 200 cells/μl to have AIDS, regardless of whether they are sick or well. The 2006 WHO recommendation for antiretroviral therapy in adults from resource-limited settings is to: (1) treat those with WHO Stage 1 or 2 disease and CD4+ T-cell count<200 cells/μL; (2) consider treatment for WHO Stage 3 disease and CD4+ T-cell count is <350 cells/μL, but initiate treatment before CD4+ T-cell count drops below 200 cells/μL, and (3) treat patients with WHO Stage 4 disease regardless of the CD4+ T-cell count (See the World Health Organization, Antiretroviral therapy for HIV infection in adults and adolescents: recommendations for a public health approach (2006).

Three CD4 T-lymphocyte categories commonly used are defined as follows:
Category 1: greater than or equal to 500 cells/μL,
Category 2: 200-499 cells/μL, and
Category 3: less than 200 cells/μL.

CD4 T-cells are not the only type of lymphocyte. Another type of lymphocytes are CD8 T-cells, which kill abnormal or infected body cells. Instead of counting the number of CD4 cells/μL, doctors sometimes assess what proportion of all the lymphocytes are CD4 T-cells. Compared with the absolute CD4 lymphocyte count, the percentage of CD4 T-cells of total lymphocytes (or CD4 percentage) is less subject to variation on repeated measurements. Data correlating natural history of HIV infection with the CD4+ percentage have not been as consistently available as data on absolute CD4+ lymphocyte counts, however, these numbers can still be useful. For pediatric patients (age<5 years), antiretroviral therapy is recommended for CD4%<15-25%, depending on the patient's age.

In HIV-negative people, a normal CD4 percentage is about 40%. A CD4 percentage below about 20% is thought to reflect a risk of opportunistic infections about the same as an absolute CD4 count of about 200 cells/μL. Some doctors argue that this is potentially the most accurate CD4 test, although it is not very sensitive to small changes.

The methods and devices described herein can be used to provide the CD4 percentage. For example, a specific binding reagent for lymphocytes can be immobilized in a separate detection zone. Anti-CD45 antibodies are one such specific binding reagent. The quantity of CD4 target cells detected in the CD4 detection zones is compared with the quantity of total lymphocytes detected in the CD45 detection zone to provide a CD4 percentage. Note that the immobilized CD45 specific binding reagent serves as both a detection zone and as a control zone. A person of ordinary skill in the art would readily be able to select other specific binding reagents for lymphocytes that can be used in this manner.

A third approach is called the CD4:CD8 ratio, in which the number of CD4 cells in a sample of blood is compared with the number of CD8 cells. The result is given as a single figure, which indicates how many CD4 cells are present for each CD8 cell. A normal result is greater than 1 (i.e. there is at least one CD4 cell for every CD8 cell in the sample), but this tends to fall to below 1 if HIV disease progresses. Both the CD4 percentage and the CD4:CD8 ratio are also affected by changes in the number of CD8 cells, which tends to rise through the course of HIV infection.

The methods and devices described herein can be used to provide the CD4:CD8 ratio. For example, a specific binding reagent for CD8 lymphocytes can be immobilized in a separate detection zone. Anti-CD8 antibodies are one such specific binding reagent, although this method would still require the whole blood to be depleted of monocytes. The quantity of CD4 target cells detected in the CD4 detection zones is compared with the quantity of total lymphocytes detected in the CD8 detection zone to provide a CD4:CD8 ratio. Note that the immobilized CD8 specific binding reagent serves as both a detection zone and as a control zone. A person of ordinary skill in the art would readily be able to select other specific binding reagents for CD8 lymphocytes that can be used in this manner and that would not require the whole blood sample to be depleted of monocytes.

These data can be used to monitor the immune system. Most people with HIV find that over time their CD4 cell count falls, although there can be long periods when it remains very stable. If it falls below certain levels, the patients are potentially at risk from certain opportunistic infections, so they can be offered treatments to try to prevent them. Likewise, monitoring the CD4 count can help a patient decide whether to start taking anti-HIV drugs, to try to prevent any further damage to your immune system.

If the patient is already taking anti-HIV drugs, the trend in a CD4 cell count can help to show how well the treatment is working. A steady increase in a CD4 cell count after starting treatment is a good sign; if a CD4 cell count is below 200 cells/mL when treatment is started, monitoring CD4 count will pinpoint when it is possible for the patient to stop taking prophylactic or maintenance treatments for opportunistic infections. Ongoing monitoring of the CD4 cell count will also provide useful information about the safety of stopping treatment, and when it might be advisable to re-start treatment.

The frequency at which a CD4 cell count test should be administered will depend on the current state of your immune system, and whether or not the patient is taking anti-HIV drugs. Among untreated people whose CD4 counts are above 500 cells/μL, tests are usually performed only every six to twelve months. At CD4 counts between 350 and 500 cells/μL, tests are likely to be performed about every three to six months, or can be more often if recent tests suggest that the CD4 count is falling. People with counts between 250 and 350 cells/μL can be offered a CD4 count more often, so that precautions such as prophylaxis against PCP (pneumonia) can be suggested if the count falls below 200 cells/μL.

The use of the device of the invention to detect CD4+ T-cells in HIV infected individuals is only one exemplary use of the device. The lateral flow device of the invention can be modified to detect any target cell of interest. Thus, it is contemplated that the detection of infection, the detection of cancerous conditions, and the detection of immune responses can be accomplished using a device of the invention.

Malaria is an infection caused by protozoan parasites of the genus Plasmodium (phylum Apicomplexa). In malaria infected individuals, Plasmodium parasites reproduce in red blood cells. As a consequence, the Plasmodium surface adhesive protein (called PfEMP1, for Plasmodium falciparum erythrocyte membrane protein 1) is exposed on the surface of red blood cells. The device of the invention can be modified to diagnose malaria in an individual by detecting malaria infected red blood cells in a whole blood sample. In this embodiment, the one or more detection zones comprise an immobilized binding reagent specific for PfEMP1. A red blood cell specific binding reagent coupled to a detectable label is then used to detect the red blood cells. Additionally methodologies used to permeabilize red blood cells while maintaining their intact configuration, and immobilizing and detecting just the malarial infected cells is also possible.

Leukemia is a cancer of the blood or bone marrow and is characterized by an abnormal proliferation of white blood cells (leukocytes). The device of the invention can be modified to semi-quantitatively detect white blood cells. In this embodiment, immobilized CD4 specific binding reagents and/or immobilized CD8 specific binding reagents can be used to capture T-cells, and immobilized CD20 specific binding reagents can be used to capture B-cells. Additional subtypes of white blood cells may be detected by immobilizing a binding reagent specific for the cell type of interest in the detection zone. Thus, the device of the invention can be used as a rapid, point-of-care test to detect elevated white blood cell counts in an individual suspected of having leukemia.

Immune responses to infection result in B-cells expressing immunoglobulins that specifically recognize antigens of the pathogen. A device of the invention can be used to detect an immune response to a specific infection. In this embodiment, an antigen expressed by the pathogen is immobilized in the detection zone. As a whole blood sample flows across the detection zone, B-cells expressing an immunoglobulin specific for the antigen are captured when the immunoglobulin binds the antigen. A B-cell specific binding reagent coupled to a detectable label is then used to detect the captured B-cells. Detection of B-cells in the detection zone and detection of IgM on the bound B cell indicates a recently acquired active immune response to the pathogen and the presence of infection. Thus, the device of the invention can be used as a rapid diagnostic test for infections. Non-limiting examples of infections common in resource limited settings include E. coli, Listeria monocytogenes, Neisseria meningitidis, Streptococcus pneumoniae, Haemophilus influenzae type b, Salmonella, and Group B streptococcus. In another embodiment, a Class I or Class II major histocompatibility multimer bound to a specific peptide (example influenza or HIV peptides) is the specific binding reagent. A whole blood sample possessing CD8 or CD4 T cells respectively that specifically recognize the complex MHC-peptide will bind to the binding reagent, indicating an immune response to an active, inactive, acute or chronic infection.

The following Examples are provided to further assist those of ordinary skill in the art. Such examples are intended to be illustrative and therefore should not be regarded as limiting the invention. A number of exemplary modifications and variations are described in this application and others will become apparent to those of skill in this art. Such variations are considered to fall within the scope of the invention as described and claimed herein.

### EXAMPLES

### Example 1

### Preparation of Non-Porous Chips

Non-porous lateral flow devices were purchased from a commercial source (4Castchip™, Amic AB, Sweden), and are generally described in U.S. Patent Publication Nos. 2005/0042766, 2006/0285996 and 2007/0266777, which are herein incorporated by reference in their entirety.

The devices contain a highly ordered array of micropillars that drive flow of liquid in an open channel by capillary action. The micropillars are substantially vertical protrusions from the surface of the substrate. The devices are structured to generate lateral flow of whole blood through the micropillars without structurally impeding the flow of leukocytes. The flow of liquid across the chip is controlled by the pillar geometry and the channel length. Preferably, the device permits flow of between about 10 μl to about 100 μl of whole blood. In one embodiment, the micropillars are spaced about 30 μm apart, measuring from the edges of adjacent micropillars.

These devices have an aldehyde functionalized surface that allows simple coupling of capture molecules to the chip, while simultaneously reducing background signal from non-specific binding. Specifically, amine groups on specific binding reagents react by a simple one-step Schiff base reaction to couple covalently to the surface of the device.

### Example 2

### Preparation of a Chip Surface for Covalent Attachment of Antibodies

Non-porous devices are coated with oxidized dextran. Oxidized dextran is prepared as described previously (See U.S. Pat. No. 5,466,609).

In a standard scaled-up preparation, 80 g of dextran (2,000,000 mw, SIGMA p/n D-5376) is transferred to 1 quart glass blender bowl containing 600 ml distilled water. The solid is blended for about 2-5 minutes at medium speed to dissolve all the dextran. After that, 8.56 g of sodium periodate (Sodium m-Periodate, SIGMA p/n S-1878) is dissolved in 100 ml of distilled water and the resulting solution is added slowly to the dextran solution over about 10 minutes using vigorous magnetic stirring. After the addition is completed, the resulting mixture is stirred at room temperature for an additional 3 hours. The resulting viscous reaction mixture is then diluted to 2 liters with distilled water and desalted using a hollow fiber cartridge. The initial specific conductance is 1.5 mmho-cm⁻¹ or higher and the initial pH is 4.0. About 18-22 liters of distilled water is used to obtain a solution having a final pH of 6.0-6.5. The final volume of washed, oxidized dextran solution is 800 ml.

A chemical analysis is performed to confirm oxidation, showing that every twelfth dextran ring is cleaved open, providing two aldehyde groups.

A hollow fiber cartridge (polysulfone, 3 ft³ membrane surface area, 1 mm diameter fibers and 5,000 MW cut-off) is mounted vertically with an input power pump (two pump heads, maximum flow rate of about 4.56 liters/minute with No. 18 Norprene™ food grade tubing) delivering 15-20 psi which corresponds to 5-10 psi in the retentate line. The filtrate is collected at 50-100 ml/min.

Oxidized dextran can be applied to the surface of the non-porous lateral flow devices, such as those described in Example 1.

### Example 3

### Preparation of Porous Membranes

Membranes are useful in the non-lateral flow embodiments described below. Generally, such membranes have a hydrophobic backbone with hydrophilic surface, a surface neutral charge, low non-specific binding, consistent pore size, thickness and protein binding capacity, optimal porosity enabling radial diffusion or non-lateral flow, low coefficient of variation (CV) for capillary rise time over shelf life, are amenable to chemical modification to enable covalent conjugation to a specific binding reagent, and have multiple functionality-conjugate application, such as sample application, reaction surface, and wicking action.

19 different membrane configurations were cast. The configurations used different concentrations of an organic polymer and wet thickness of the cast film. They were cast as supported or unsupported, where the membranes were supported by non-woven spun fabric such as Hollytex® (Ahlstrom Filtration Co.) or Whatman paper filter. The castings were performed using coagulation baths at various temperatures, such as at 4° C., room temperature, 60° C., and 75° C., however, the use of alternative temperatures is also contemplated. Different solvent concentrations were used; the concentrations ranged from 100% of each solvent to a combination of solvent/non-solvent. The non-coagulated organic polymer cast films were dried in a convection oven.

In one embodiment, a Polyacrylonitrile membrane was prepared. 12-14% Polyacrylonitrile (PAN) (150,000 mw, Polysciences Inc. p/n 03914) by weight and in powder form was dissolved in N,N dimethylformamide (DMF) (VWR, p/n DX1730-3) using a blender (Commercial Blender, Waring, p/n 34B297). The polymer solution was allowed to reach room temperature and de-aerate. Glass plates (8″×12″ glass plates with ground edges) were cleaned with paper towels and acetone to remove any fatty deposits.

A stainless steel tray (VWR, p/n 62687-049) was filled with deionized water. The tray was placed on a hot plate to heat the deionized water to approximately 70° C. to 75° C. The casting knife blade opening (Elcometer, p/nK0003580M005) was adjusted between 250 μm and 375 μm. The PAN/DMF solution was poured slowly onto the top edge of the glass plate, avoiding pouring the air bubbles that can be mixed into the solution by pouring. The solution was drawn towards the pourer to form a liquid polymer film on the surface of the glass plate. The cast film was slowly immersed (head forward) at an angle into the hot deionized water, avoiding the formation of waves. The membrane was allowed to coagulate for about 5 minutes. The coagulated membrane was removed from the hot water and rinsed in deionized water at room temperature.

9 different membranes were evaluated using 0.1% Methyl orange solution in deionized water and EDTA-treated whole blood from normal donors. One 14% Polyacrylonitrile membrane was generated as described as above in a 75° C. coagulation bath, which had a 375 μm wet thickness. The results using this membrane are shown below in Table 2.

TABLE 2
Polyacrylonitrile Membrane

| Volume | | | | | | | |
|---|---|---|---|---|---|---|---|
| added to | | 0.1% Methyl | | Donor 1 | | Donor 2 | |
| membrane | | Orange in D/W | | Whole Blood | | Whole Blood | |
| 10 μl | Time | 1 | seconds | 5 | seconds | 60 | seconds |
| | Diffusion | 10 | mm | 8-10 | mm | 8-10 | mm |
| | diameter | | | | | | |
| 50 μl | Time | 5 | seconds | 10 | seconds | 120 | seconds |
| | Diffusion | 23-24 | mm | 21-22 | mm | 21-22 | mm |
| | diameter | | | | | | |

It was determined that at the same concentration of organic polymer, unsupported membranes had better lateral porosity as opposed to supported membranes. Additionally, membranes that had been coagulated at higher temperatures show improved lateral porosity. Membranes with consistent radial flow were identified by whole blood migration. It was determined that regardless of donor, the radial diffusion diameter correlated with the volume of liquid placed on membrane, however, the time required to reach the radial diffusion diameter varied depending on liquid media as well as the donor.

### Example 4

### Modification of Membrane Surfaces

The PAN membrane surface described in Example 3 contains nitrile groups (—CN) and is hydrophobic by nature. In order to introduce a reactive functional group, the membrane surface needs to be chemically modified. It is desirable to keep the surface hydrophilic after the chemical modification to prevent the hydrophobic adsorption of non-targeted proteins onto the membrane surface.

The first step was the controlled hydrolysis of the nitrile groups, to form amido groups (—CONH₂).

3.0 liters of a hydrophilization bath was prepared in a heavy duty 4.0 liter beaker, (VWR, p/n 89000-230). 0.1 M EDTA (EM, p/n EX0539-1), 0.3 M fumaric acid (Mallinkrodt, p/n 0898-59), 0.025 M Sodium tetraborate decahydrate (Borax, J T Baker, p/n 3570-01) were dissolved in 2.5 liters of deionized water. The solution was heated to ˜50-55° C. using a hot plate stirrer and stirring bar. 0.3 M hydrogen peroxide (H₂O₂) (VWR, p/n VW3742-1) was added. The pH was adjusted to 9.0 with sodium hydroxide pellets (EMD, p/n SXO590-1), using a pH meter. The final volume of the lyophilization bath was brought to 3.0 liters with deionized water.

The cast PAN membranes were immersed into the hydrophilization bath between a beaker wall and a PVC spacer pipe containing side holes for proper liquid circulation. The membranes were hydrophilized for about 2 hours to 4 hours, depending on the desired degree of hydrophilicity. They were then removed from the bath and washed in deionized water at room temperature.

A functional group was then introduced on the surface of the membrane by converting the obtained amido groups (—CONH₂) to hydrazo groups (—NH—NH₂).

A 3 M solution of hydrazine hydrate (NH₂—NH₂) (SIGMA, p/n 207942) in deionized water was prepared in the fume hood. The hydrophilized PAN membranes were immersed into the hydrazine solution and allowed to react for 4 hours at room temperature. The hydrazo surface modified membranes were washed in deionized water.

A Schiff base was formed between the membrane surface hydrazo groups and glutaraldehyde. A 5% glutaric dialdehyde (GA) (SIGMA, p/n G4004) solution was prepared in deionized water or in pH 9.2 borate buffer in the fume hood. The hydrazine modified membranes were immersed into the GA solution, and were allowed to react for 4 to 24 hours at room temperature. The membranes were then washed extensively with deionized water until no free GA odor was detectable from the membrane surface.

The membranes were tested for the presence of surface aldehyde groups with the Schiff reagent. The depth of the developed purple color was an indication of the degree of Schiff base formation.

The water in the pores of the membranes was replaced with acetone by soaking the membranes in acetone (VWR, p/n AX 0120-3) for 5 minutes in the fume hood. Membranes were air dried at room temperature under weight, placed between clean paper towels to prevent shrinkage and warping. The final product was stored in the refrigerator at 4° C. between paper towels in closed Ziploc bags.

Alternatively, the surfaces of commercial membranes are modified. Whatman S14 and S17 and Porex® X4588 (Porex Corporation) were modified using the following procedure. The S14 and S17 materials are non-woven, spunbound glass fibers, kept together with polymeric binders, while Porex is a microporous polyethylene sheet, having surface carboxyl groups introduced by the manufacturer. The surface modification of these membranes involved the formation of polymeric glutaraldehyde within the body of the material. A mixture of 25% of glutaraldehyde and concentrated sulfuric acid (98%, EMD, p/n SX1244-5) was used in a 3:1 molar ratio to catalyze the polymerization of GA, forming cyclic trimers and linear polymers. The process was done at room temperature for 4 to 24 hours in the fume hood. The commercial membranes were then thoroughly washed from the reactants and tested for the presence of aldehyde groups with the Schiff Base.

After these procedures, the specific binding reagents such as antibodies can be covalently bound to the membranes.

### Example 5

### Protocol for Antibody Capture of Cells on Chips

Materials
Cyto-Trol™, Beckman Coulter part no. 6604248, lot no. 731902K.
Cyto-Trol™ control cells, Beckman Coulter part number 6604249, lot number 729113. This lot has 2545±91 CD4 cells/0.1 mL or 25,450±910 CD4 cells/vial.
Reconstitution Buffer, Beckman Coulter part number 6604250, lot number 731879.
PE-labeled CD3 antibody, CD3-RD1, Beckman Coulter, part no. 6604627, lot no. 745919F; 0.5 mL, 100 tests, 5 μL per test, 2.0 μg CD3 antibody per test.
Monoclonal anti-human CD4, mouse IgG 1, Beckman Coulter, Clone SFCI 121 T4D 11 (T4), 3.371 mg/mL in PBS+0.1% NaN3; 500 μL, Jul. 12, 2007. This is a concentrate of T4 antibody (part no. 6602138) without bovine serum albumin.
Monoclonal mouse IgG 1 isotype, Beckman Coulter, 2 mg/mL in PBS+0.1% NaN3. This is a concentrate of mouse IgG1 isotype (part no. IM0571) without bovine serum albumin.
Amic chips, B2.2, item no. 513.
100 mM Sodium borate buffer, pH 9.0 with 2% sucrose.
50 mM Sodium phosphate buffer, pH 7.4, 150 mM NaCl, 0.25% (v/v) Tween 20, 0.05% (w/v) NaN3, 0.5% (w/v) Pluronic F108.
Dulbecco's phosphate buffered saline (PBS), without calcium chloride or magnesium chloride, Gibco, part no. 14190, lot no. 1362579.
Centrifuge tube, polypropylene. 0.5 mL, Sarstedt, catalog number 72.730.006.
Centrifuge tube, polypropylene. 1.5 mL, Sarstedt, catalog number 72.692.005.
Polyethylene disposable transfer pipette, non-sterile, 4 mL capacity, 15.5 cm length, VWR part no. 16007-178.
Scotch brand double sided tape, permanent, 1.25 cm wide.

Methods

Anti-human CD4, clone SFC1121 T4D 11 (T4) was prepared. The 3.371 mg/mL T4 antibody was diluted to 1 mg/mL with 100 mM sodium borate buffer, pH 9.0 with 2% sucrose. For four chips at 10 μL per chip, 40 μL was required, and 46.5 μL was prepared. 13.8 μL of concentrated T4 antibody was added to 32.7 μL 100 mM sodium borate buffer, pH 9.0 with 2% sucrose.

Monoclonal mouse IgG1 isotype was prepared. The 2 mg/mL mouse IgG1 isotype antibody was diluted to 1 mg/mL with 100 mM sodium borate buffer, pH 9.0 with 2% sucrose. For four chips at 10 μL per chip, 40 μL was required, and 50 μL was prepared. 25 μL of concentrated mouse IgG1 antibody was added to 25 μL 100 mM sodium borate buffer, pH 9.0 with 2% sucrose.

Buffer blanks and antibody solutions were applied onto Amic chips at the 1.0 cm mark of the detection zone, which is about at the half way point in this zone. 10 μL of buffer only (100 mM sodium borate buffer, pH 9.0 with 2% sucrose) was applied to two chips using a P20 Pipetman®. These chips did not have capture antibody. A 10 μL aliquot of diluted anti-human CD4 (1 mg/mL) in 100 mM sodium borate buffer, pH 9.0 with 2% sucrose was applied to four chips using a P20 Pipetman®. This was 10 μg of total antibody protein. A 10 μL aliquot of diluted monoclonal mouse IgG1 isotype (1 mg/mL) in 100 mM sodium borate buffer, pH 9.0 with 2% sucrose was applied to four chips using a P20 Pipetman®. This was 10 μg of total antibody protein. The antibodies and buffers were applied by positioning the Pipetman® so it was parallel to the length of the chip and pointing towards the sample zone. With the pipette tip just above the surface of the chip, the liquid was dispensed slowly and allowed to touch the surface of the chip. The liquid was continued to be dispensed slowly without allowing the pipette tip to touch the chip surface. After dispensing the liquid, the pipette tip was lifted straight up.

The control and antibody chips so striped were then incubated. The chips were placed in a covered box in the refrigerator at 4-8° C., overnight (about 16-20 hours) with >60% humidity. Humidity was provided by cutting a Kimwipe® in half and dabbing each piece with another Kimwipe® that has been soaked with water. The two half pieces of Kimwipe® were placed in the box with the chips. After incubation in the refrigerator, the chips were removed. The width of the applied antibody or buffer only on each chip was measured and recorded.

Cellulose wicks were then prepared, and the chips were placed on slides. 1 mm thick blotting paper was cut into 7 mm wide by 14 mm long strips. A 2.5×5 cm polyethylene sheet was mounted to the end of the absorbent zone of each of the chips using double sided tape. A cellulose wick was applied to the top of the double sided tape so one end was in the absorbent zone. The wick was wet with 2-3 drops of water using a polypropylene disposable pipette, taking care not to over-saturate the wick. A second cellulose wick was added on top of the first wick, without adding any more water. When the two wicks were lightly pressed, no excess water appeared. If water appeared, the top wick was changed without adding more water.

The chips were then treated with Pluronic F108 and Tween 20 blocking-wetting reagent. 50 mM Sodium phosphate buffer, pH 7.4, 150 mM NaCl, 0.25% (v/v) Tween 20 (polyoxyethylene (20) sorbitan monolaurate), 0.05% (w/v) NaN3 and 0.5% (w/v) Pluronic F108 was prepared. Three 20 μL aliquots of this buffer were added to the chip: one aliquot in the sample zone, one in the front of detection zone and one at the end of the detection zone. Each aliquot was dispensed slowly, so that it was not applied to just one spot. After application of the blocking-wetting reagents, the chips sat covered for 30 minutes at room temperature.

The Cyto-Trol™ cell sample was prepared. Cyto-Trol™ lot no. 729113 had 25,450±910 CD4 cells/vial. Twenty-fold concentration results in 509,000+18,200 CD4 cells/mL. The vials of Reconstitution Buffer and the vials of Cyto-Trol™ were allowed to come to room temperature. 1.0 mL of Reconstitution Buffer was added to each vial of Cyto-Trol™. The vials were mixed by finger flick and swirling. The vials were allowed to stand at least 10 minutes at room temperature. The contents of each vial were transferred into one 1.5 mL polypropylene centrifuge tube, and centrifuged at 500×g at 20° C. in ProteomeLab SP with SX4250 rotor and adapter part no. 368336. The supernatant was aspirated with a P20/200 pipette tip on tubing. Each cell pellet was resuspended in 50 μL PBS with finger flick mixing. The two 50 μL PBS cell suspensions were combined into one 0.5 mL centrifuge tube, thus providing a 20-fold concentrate of Cyto-Trol™. The 20-fold concentrate of Cyto-Trol™ was diluted two-fold to a 10-fold concentrate by combining 25 μL of the 20-fold concentrate of Cyto-Trol™+25 μL of PBS in a 0.5 mL centrifuge tube, and mixed by finger flick and swirling.

Either 2500 or 5000 CD4 Cyto-Trol™ cells were added to each Amic chip. 10 μL of the 20-fold concentrate of Cyto-Trol™ cell sample was added to the sample zone of two control chips, two CD4 antibody chips, and two mouse IgG1 isotype chips. The calculated cell count was 5,084 CD4 cells per 10 μL. 10 μL of the 10-fold concentrate of Cyto-TrolTM™ cell sample was added to the sample zone of two control chips, two CD4 antibody chips and two mouse IgG1 isotype chips. The calculated cell count was 2,542 CD4 cells per 10 μL. The cell sample was allowed to react with the chip for 5 minutes at room temperature.

All the chips were washed with two 20 μL aliquots of PBS in the wash zone, waiting for 3-5 minutes or until the PBS completely flowed into the wick. This removed the non-CD4 cells that can bind the PE-labeled CD3 antibody.

The diluted PE-labeled CD3 antibody, CD3-RD1, was prepared and added to the Amic chips. CD3-RD1 was diluted four-fold with PBS. To make 220 μL, 165 μL of PBS was added to 55 μL of CD3-RD1. 20 μL of the four-fold diluted CD3-RD1 was added to the sample zone of each chip. The chips were allowed to sit for five minutes covered in the dark at room temperature. Six 20 μL aliquots of PBS were added to the wash zone of each chip in total; three 20 μL aliquots of PBS were added at one time to each chip. This was to remove the unbound labeled antibody. Chips were kept in the dark during the washes. After the last PBS wash, a 20 minute waiting period was provided for the solution to migrate to the wick and dry the chip. If the flow was slow, the top wick was changed to a fresh dry wick.

One of the chips was then scanned at a 30 micron resolution with the Scan Array Lite (Perkin Elmer) to see if there was a fluorescent signal in the middle of the detection zone. If there was fluorescence, the experiment worked.

Each slide was then scanned with the Scan Array Lite at 10 micron resolution for analysis with the ImageJ software (public domain Java image processing program available at http://rsb.info.nih.gov/ij/index.html).

Each slide was examined and photographed with a video microscope at 1×, 2×, and 3× magnification.

### Example 6

### Protocol for Antibody Capture of Cells on Chips Using a Humidified Chamber

For multi-stripes of anti-human CD4 antibody or mouse IgG1, a P2 Pipetman® was used to stripe. The CD4 antibody or mouse IgG1 was striped at 6 positions per chip in the detection zone at 0.5 microliter/stripe of 5 mg/ml of corresponding antibody preparation.

Materials
Humidified Chamber
Pluronic blocking buffer [Pluronic F108 0.5%, sodium chloride 150 mM, Tween-20 (polyoxyethylene (20) sorbitan monolaurate) 0.25%, and Sodium Azide 0.05%, in 50 mM phosphate buffer, pH 7.4.
Wick, 15 mm×10 mm filter paper (Extra thick filter paper, cat. # 1703960, lot # PLN100291, Bio-Rad Laboratories, CA, USA)
PBST (PBS plus 0.25% Tween-20 (polyoxyethylene (20) sorbitan monolaurate))
Whole blood, CD4+ cells- or monocyte-depleted preparations, or plasma

Preparation of the Humidified Chamber

The humidified chamber was made from a slide holder that can hold 5 slides, with two holes 5 mm in diameter. One hole is near the wash zone and allows addition of wash solution to the chip and one hole is near the sample receiving zone and allows for the addition of the sample to the chip. There was also a rectangular opening of 15 mm×10 mm near the waste zone at the end of the slide holder for placing the wicks onto the chip. A piece of filter paper 75 mm×20 mm was placed inside the chamber. 4 ml water was added to the filter paper.

Procedure for Chip Development Inside the Humidified Chamber

A single chip was placed into the middle level of the chamber, such that the wash and sample receiving zones line up with the 5 mm holes in the humidified chamber. The chip was blocked by applying 50 μl of Pluronic blocking buffer to the wash zone through the hole in the humidified chamber. The buffer was allowed to laterally flow to the waste zone. The chip was incubated for 30 minutes at room temperature inside the humidified chamber.

Two pre-wetted wicks (15 mm×10 mm, stacked) were placed at the end of the chip at the waste zone through the rectangle opening in the humidified chamber. The chip was washed by addition of 50 μl of PBST to the wash zone through the hole on the humidified chamber. The chip was allowed to incubate for 5 minutes at room temperature inside of the chamber. If the chips became dry in the course of running the assay, one drop (˜10 μl) of PBST was added and allowed to flow into micropillars before proceeding.

10 μl of a whole blood sample was added to the sample zone on the chip through the hole on the humidified chamber. After the sample had moved by lateral flow into the micropillars the chip was washed by adding one drop (˜10 μl) of PBST to the wash zone. After this first wash solution had moved into the micropillars, two more washes were done by adding 25 μl PBST to the wash zone, each time waiting until the previous wash solution had moved into the micropillars. Chips were allowed to incubate at room temperature for 5 minutes.

Target cells were labeled by applying one drop (˜10 μl) of black beads (lot 12-BK-2.2K.1, Invitrogen) coupled to a target cell specific binding reagent to the sample zone. If necessary, a second drop of black beads coupled to a target cell specific binding reagent can be applied to the sample zone. Chips were subsequently washed 1-2 times by adding one drop (˜10 μl) of PBST to the wash zone.

### Example 7

### Results of Antibody Capture of Cells on Chips

The results of the experiments described in Example 5 are summarized in Table 3.

TABLE 3

| | Antibody/ | Post | | | Microscopic |
|---|---|---|---|---|---|
| Capture | Buffer | Block | Replicate | Scan Array | Evidence of |
| Antibody | Width (mm) | Wash | No. | Fluorescence | Cells |
| None | 10 | yes | 1 | − | − |
| | 10 | yes | 2 | − | − |
| | 10 | no | 1 | − | − |
| | 15 | no | 2 | − | − |
| CD4 | 9 | yes | 1 | + | + |
| | 7 | yes | 2 | + | + |
| | 7 | no | 1 | + | + |
| | 8 | no | 2 | − | ? |
| mIgGI | 5 | yes | 1 | − | − |
| isotype | 5 | yes | 2 | − | − |
| | 5 | no | 1 | − | − |
| | 5 | no | 2 | − | − |

Both fluorescence analysis and video microscopic examination indicated that with the CD4 antibody applied to the Amic chip, three of the four chips with the CD4 antibody bound Cyto-Trol™ cells. The fluorescence was weak and only appeared one or two fluorescent lines appear on the scan (FIGS. 8A-C), however, this area with fluorescence corresponded exactly to the position where the cells were observed on the same chips by video microscopic examination (FIGS. 9A-F). The antibody was applied about 4 mm upstream from the capture region and diffused evenly up and downstream. The cells were captured by the CD4 antibody on the first one-to-two rows of micropillars on the chip to where the antibody diffused.

### Example 8

### pH Dependence

Anti-CD4 antibody was applied as described in Example 5, except the pH of the buffer was varied to determine the optimal pH for covalent attachment to the non-porous surface of the chip. The pH levels tested were 7.4, 8.5, and 9.0.

1000 CD4 Cyto-Trol™ cells were added to each Amic chip as described in Example 5, and were visualized with diluted PE-labeled CD3 antibody, CD3-RD1. The chips were then scanned at a 30 micron resolution with the Scan Array Lite (FIGS. 10 A-E). Better signal was noted at a lower concentration of capture antibody at the higher range of the pHs tested.

### Example 9

### Detecting Varying Cell Numbers

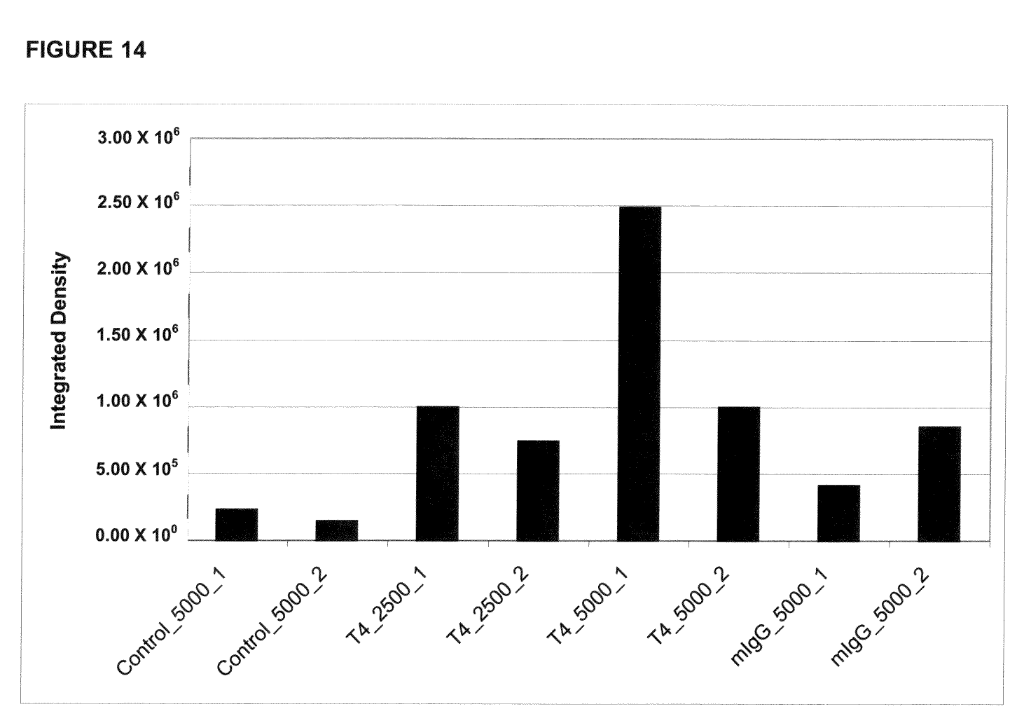

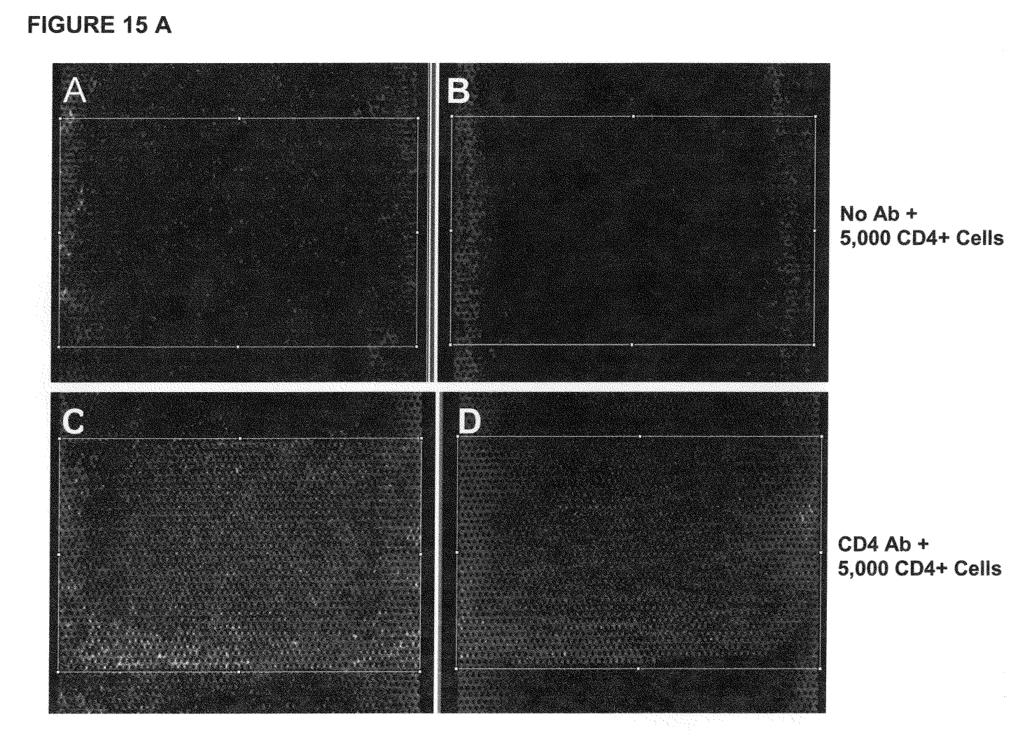

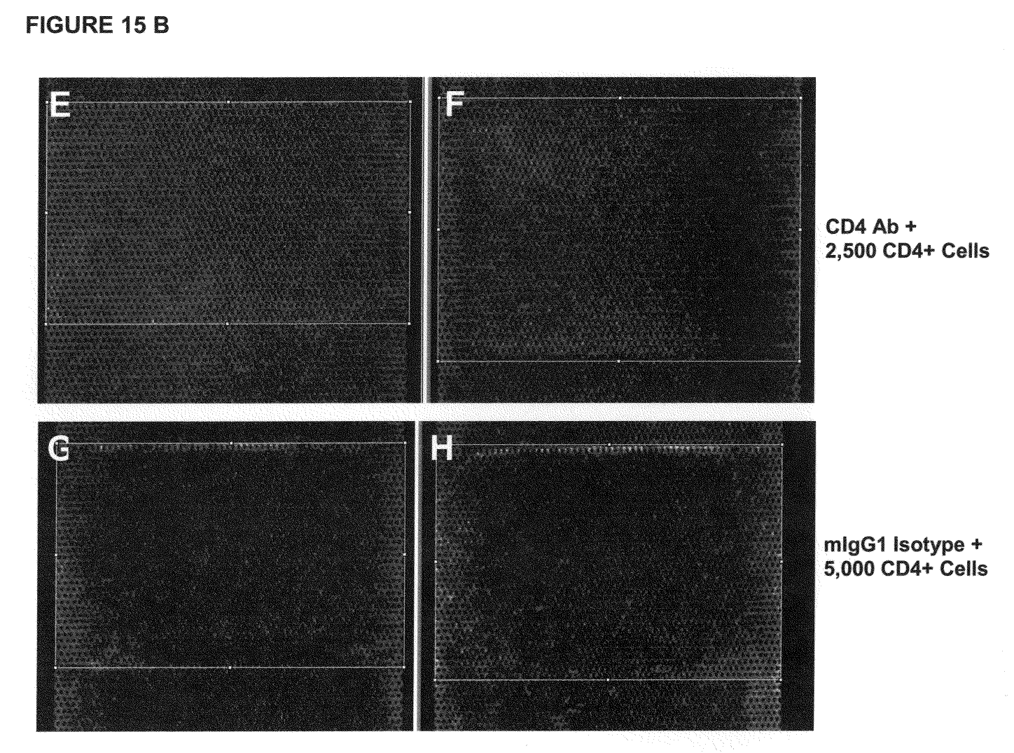

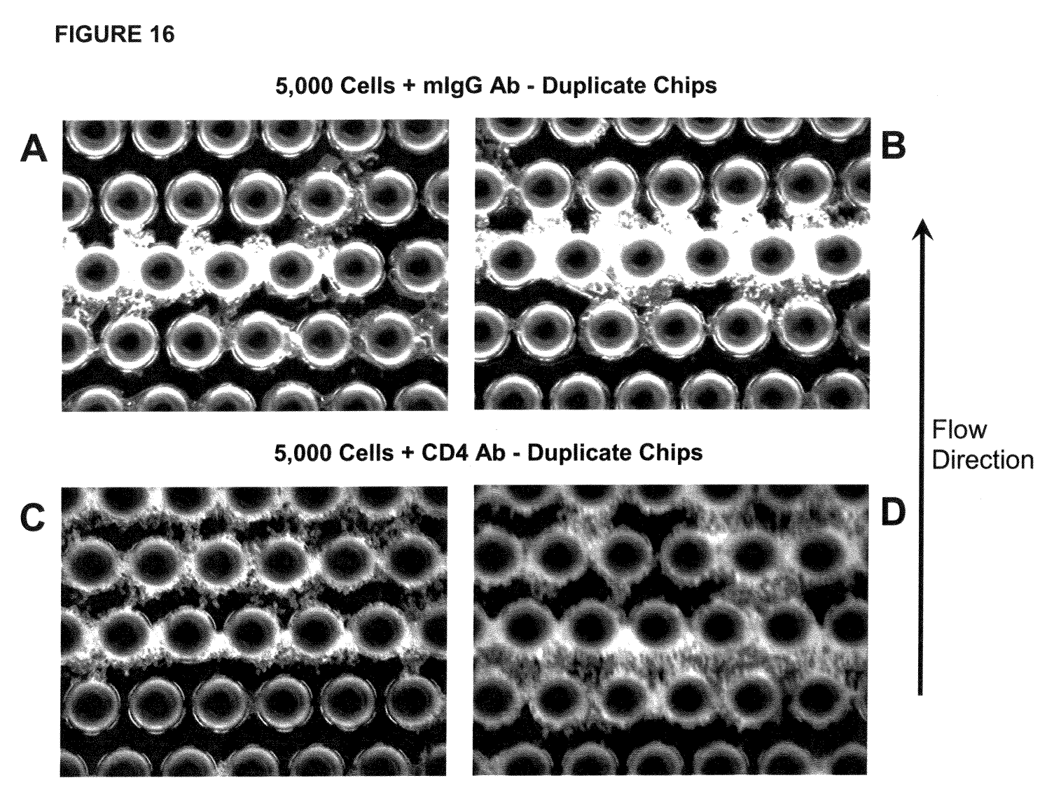

As described in Example 5, the number of cells applied to the chips was varied. In a further experiment performed as described in Example 5, both 2500 and 5000 CD4 cells were detectable with the chips, as demonstrated in FIGS. 14, 15A-H, and 16A-D.

The intensity of the detection is quantified, and is found to correspond to the number of cells captured by the detection zone.

### Example 10

### Preparation of CD4 Black Detector beads

1. Coupling of 1,2 Diaminopropane (DAP) to the Aldehyde/Sulfate Black Polystyrene Beads

Start with 10 ml of black beads, lot 12-BK-2.2K.1 (Invitrogen), at 2.1% solids. Centrifuge 10 ml of beads at 2700 rpm, for 10 minutes and re-suspend to 4.2% solids with deionized water. Then vortex and sonicate for 30 seconds to breakup aggregates. Add DAP, 16.8 mg per ml of 4.2% bead suspension, vortex and sonicate for 30 seconds. (DAP volume: (16.8×5)/10000×0.99×0.888=95.6 μl) Mix on a roller for 48 hours.

2. Reduction of the Schiff Base and Un-Reacted Aldehyde Groups on the Surface of Beads

Start with 10 mg of solid sodium borohydride per 1 ml of bead suspension. (Weight of borohydride: 10 mg/ml×(5 ml+0.096 ml)/1000 mg/g×0.98=52 mg) Add 52 mg of the solid borohydride into the tube with beads and DAP, then vortex and sonicate for 30 seconds. Mix the tube on a roller for 3 hours with periodic sonication and vortexing for 30 seconds. Release accumulated hydrogen gas during the second and third hour of the Schiff base reduction. Finally, wash 5 times with 1×PBS (pH 7.2), using centrifugation at 2700 rpm for 10 minutes and re-suspend to 5.0 ml with 1×PBS.

3. 2-iminothiolane (IT) Activation of T4 Antibody

Start with 1 mg of T4 antibody (51.99 mg/ml lot 700307CO) per 1 ml of 4.2% bead suspension. (Volume of T4 (V_{T4}): 7 mg/51.99 mg/ml=0.135 ml) The concentration of T4 during activation must be 15 mg/ml: Vₜₒₜ=7 mg/15 mg/ml=0.467 ml. Dissolve 2 mg of IT (SIGMA, lot 025K1299) in 1 ml of 1×PBS. The activation ratio between IT and Ab should equal 15:volume of IT solution: V_{IT}=7×15×0.86/2 mg/ml×1000 mg/g=0.045 ml (45 μl). The volume of PBS solution needed: V_{PBS}=Vₜₒₜ−(V_{T4}+V_{IT})=0.467 ml−(0.135 ml+0.045 ml)=0.287 ml. Then add 0.287 ml of 1×PBS into 0.135 ml of T4 solution, add 45 μl of IT solution and roller mix at room temperature for 1 hour. Pack a G50 column and equilibrate with 1×PBS (V_{G50}=0.467 ml×30=14 ml). Set up a UV monitor at 2 mm/min paper speed and scale at 1.0 absorbance units. Pass IT activated T4 through the G50 column, collect fractions and scan on a spectrophotometer at λ=280 to determine the concentration of T4 (C_{T4}, mg/ml)

4. Sulfo-SMCC Activation of Aminated Beads

Dissolve 10 mg of Sulfo-SMCC (BioSciences, lot 061406) in 1 ml of 1×PBS. Use 14.175 μl of this solution per 1 ml of bead suspension. (Vsmcc=14.175 μl/ml×5.0 ml=71 μl of Sulfo-SMCC solution) Add 71 μl of the Sulfo-SMCC solution into the tube with beads, sonicate and vortex for 30 seconds, then roller mix for 1 hour with frequent brief sonication. Finally, wash 5 times with 1×PBS using centrifugation at 2700 rpm for 10 minutes and re-suspend to 4.0 ml with 1×PBS.

5. Conjugation of Sulfo-SMCC Activated Beads and 2-iminothiolane Activated T4 Antibody

Total reaction volume during conjugation is Vₜₒₜ=5.0 ml

Concentration of T4 antibody during conjugation is Cₜₒₜ=0.700 mg/ml

Bead volume is V_{beads}=3.0 ml (7.0%).

Bead surface area: 2.7 m²/g×0.21 g=0.567 m²

Surface density of conjugated T4: Dₛ=C_{surf}×Vₜₒₜ/S_{beads}, mg/m²

TABLE 4

| | Spectro- | Volume of | | Spectro- | |
|---|---|---|---|---|---|
| | photometric | T4 needed | Volume, ml of | photometric | |
| | concentration | for | 1 × PBS needed | concentration | Surface |
| Antibody | of T4 after | conjugation | for conjugation: | of T4 in | concentration of T4, |
| name | G50 column | V_{T4} = Cₜₒₜ × | V_{PBS} = | supernatant, | mg/ml |
| and lot # | C_{T4}, mg/ml | Vₜₒₜ/C_{T4}, ml | 5 − (V_{T4} + V_{beads}) | Cₛₙ, mg/ml | C_{surf} = Cₜₒₜ − Cₛₙ |
| T4 | 1.8546 | 1.887 | 0.120 | 0.368 | 0.332 |

Add the calculated volume of PBS, VPBS (0.120 ml) into the beads, add the calculated volume of T4-IT, VT4 (1.887 ml), sonicate for 30 seconds and roller mix for 2 hours with frequent, brief sonication during the first hour of conjugation. At the end of the conjugation, centrifuge the beads at 2700 rpm for 10 minutes and remove 1 ml of supernatant. Filter the removed supernatant through the 0.2 μm AcroDisc® filter (Pall Corporation) and measure the concentration of T4 in the supernatant (Tₛₙ, mg/ml). Then, calculate the bound T4 surface density, mg/m²:

Dₛ=C_{surf}×Vₜₒₜ/S_{beads}, (mg/m²)

(0.332 mg/ml×5.0 ml)/0.567 m²=2.93 mg/m²

This value will be monitored and compared for reproducibility from lot-to-lot, as a “quality control” of the conjugation process. The range shall be established as several lots of beads are produced.

6. Blocking

After conjugation, block the unreacted maleimido groups of Sulfo-SMCC with a 5 mg/ml solution of L-cysteine for 15 minutes (SIGMA, lot 114FO672, 5 mg of L-cysteine in 1 ml of PBS). Block unreacted thiol groups of 2-iminothiolane with 20 mg/ml solution of iodoacetamide (SIGMA, lot 75H5058, 20 mg of iodoacetamide in 1 ml of PBS) for 30 minutes.

TABLE 5

| | L-cyst. ml | | Iodoacetamide, ml | |
|---|---|---|---|---|
| Vₜₒₜ, ml | Vₜₒₜ × 0.12 | Vₜₒₜ, ml | Vₜₒₜ × 0.12 | Vₜₒₜ, ml |
| 4.0 | 0.48 | 4.48 | 0.54 | 5.02 |

7. Washing

Wash beads 3 times with 5 ml of 0.2% BSA solution in PBS with 0.1% sodium azide (NaN₃), using centrifugation at 3000 rpm for 10 minutes. Leave beads in the refrigerator over night. The next day, roller mix the beads for 1 hour and wash 3 more times as before.

### Example 111

### Specificity of CD4+ T-cell Binding to the Capture antibody on the Non-Porous Micropillar Chips

1. Evaluation of Cross Inhibition Capabilities of Unlabeled CD4 T Cells to Labeled CD4+ T-Cell Binding to the T4 Zone.

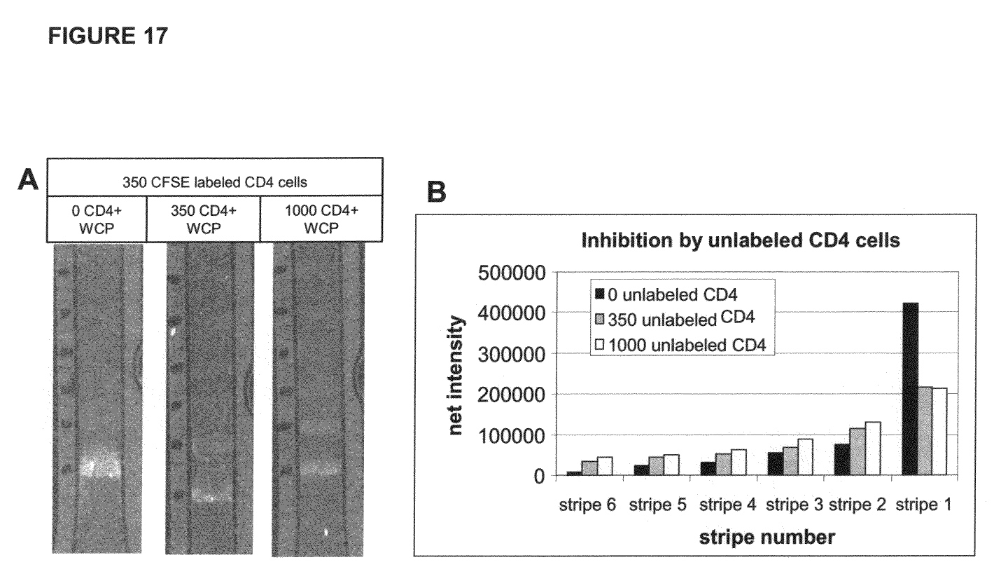

CD4+ T-cells were purified from peripheral blood mononuclear cells (PBMCs), labeled with CFSE (carboxyfluorescein succinimidyl ester; Invitrogen) and used to spike white blood cell pools (WCPs) to a concentration of 350 labeled CD4+ T-cells/μl. The binding of labeled cells to the micropillar slides was then concomitantly evaluated in the presence of unlabeled CD4+ T cells. See FIG. 17. The presence of unlabeled CD4+ cells inhibited the binding of labeled CD4+ cells to the 1st stripe while marginally enhancing the binding of labeled cells to subsequent stripes. The combination inhibition/enhancement seen in the microfluidic format for lateral flow on the slide underscores the complexity of specific capture in this assay.

b) Evaluation of Cross Inhibition Capabilities of Monocytes to CD4+ T-Cell Binding to the T4 Detection Zone.

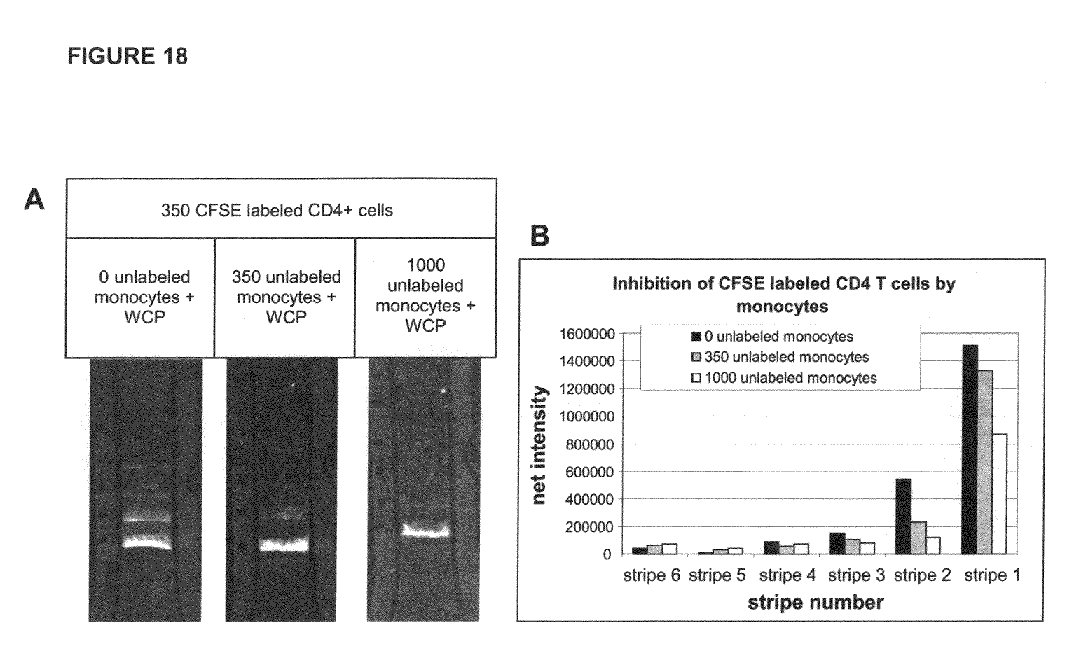

CD14+ monocytes also possess CD4+ antigen on their surface. To determine the effect of monocytes on the assay and the necessity of depleting monocytes, a similar assay was run as above with unlabeled monocytes. The assay was run 2 separate times using 2 separate donors. See FIG. 18.

As seen with CD4+ unlabeled cells, presence of monocytes did inhibit the binding of labeled CD4+ cells to the stripes when spiked into white blood cell pools. The data underscores the need to deplete monocytes to prevent false positives and also points to the specificity of CD4+ T-cell binding to the capture antibody on the slide.

c) Evaluation of Cross Inhibition of Soluble Recombinant CD4 Protein to the T4 Detection Zone

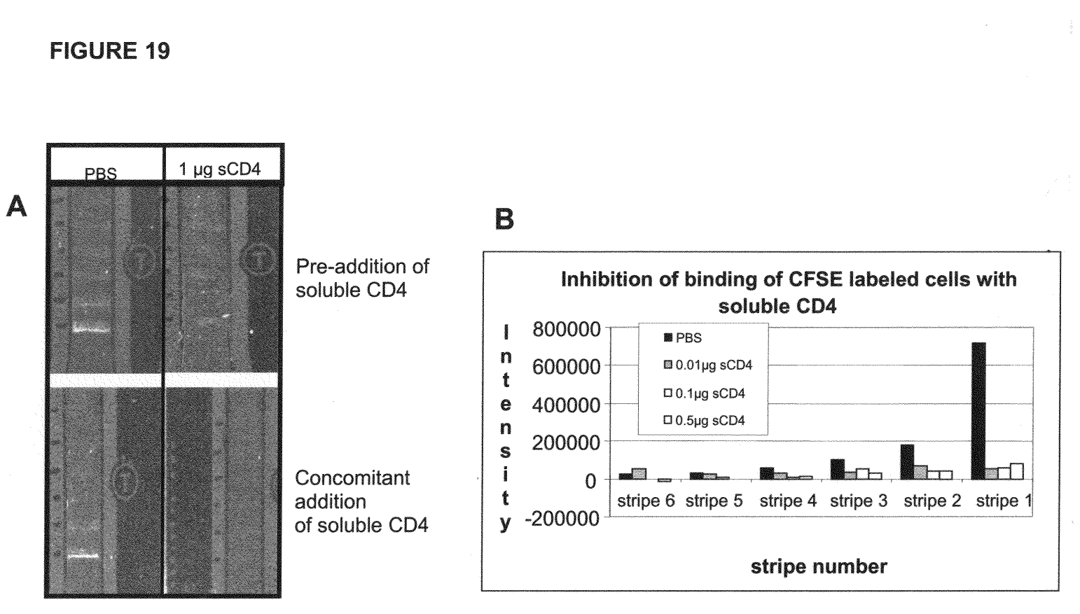

As with whole CD4+ T cells or monocytes, the effect of soluble CD4 protein (recombinant) on binding of labeled CD4+ T cells was evaluated in the multi-stripe assay either in a pre-incubation or concomitant incubation format. See FIG. 19.

Soluble CD4 at 100 fold titration (0.01->1 μg) was able to significantly impact the binding of labeled CD4+ cells to the T4 capture antibody indicating the specificity of CD4+ T-cell binding to the capture antibody on the slide.

d) Evaluation of CD3 Labeled Beads in Assay as Detector

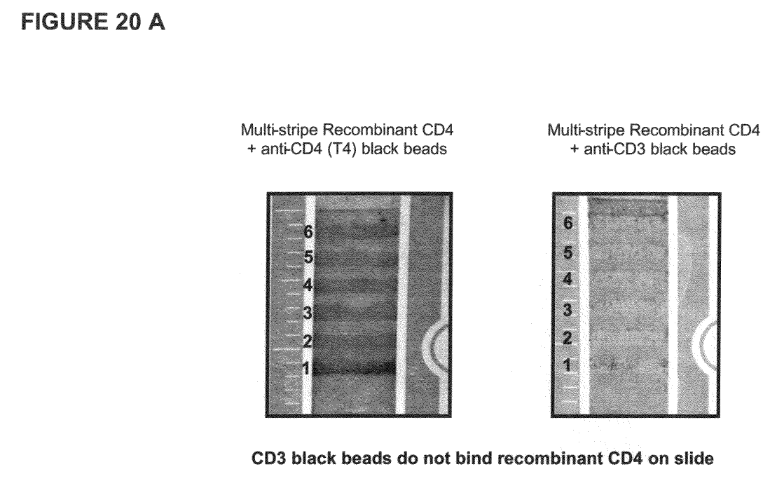

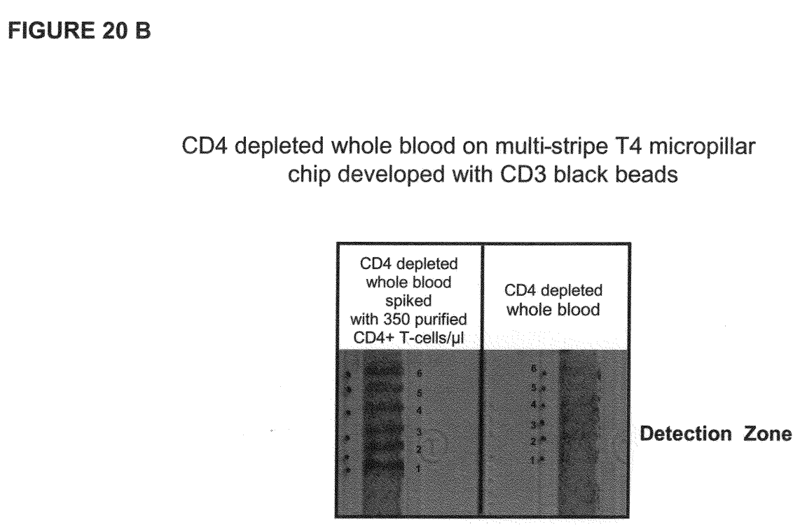

CD3 antibody conjugated to 1 micron black beads (Invitrogen) was evaluated as a detector reagent. See FIGS. 20 A and B. Recombinant CD4 was striped onto slides and incubated with anti-CD4 (T4) black beads or anti-CD3 black beads. Anti-CD3 black beads did not significantly bind to the recombinant CD4 stripes. See FIG. 20 A. CD4 depleted whole blood (with or without CD4 T cells spiked back) was captured on multi-T4 striped chips. Captured cells were labeled with anti-CD3 black beads. See FIG. 20B. CD8+CD3+ cells in CD4 depleted whole blood do not significantly bind to the T4 stripes. Post-capture labeling of CD4+ cells on the micro-pillars enables better detection compared to pre-capture labeling of cells. The results demonstrate the specificity of the CD3 labeled beads and also points to the specificity of CD4+ lymphocyte binding to the T4 capture zone due to the negative readout seen in CD4 depleted whole blood despite the presence of CD8+CD3+T cells.

### Example 12

### Integrity of the Cells Added to the Micropillar Slides after Flow Across the Micropillar Slides

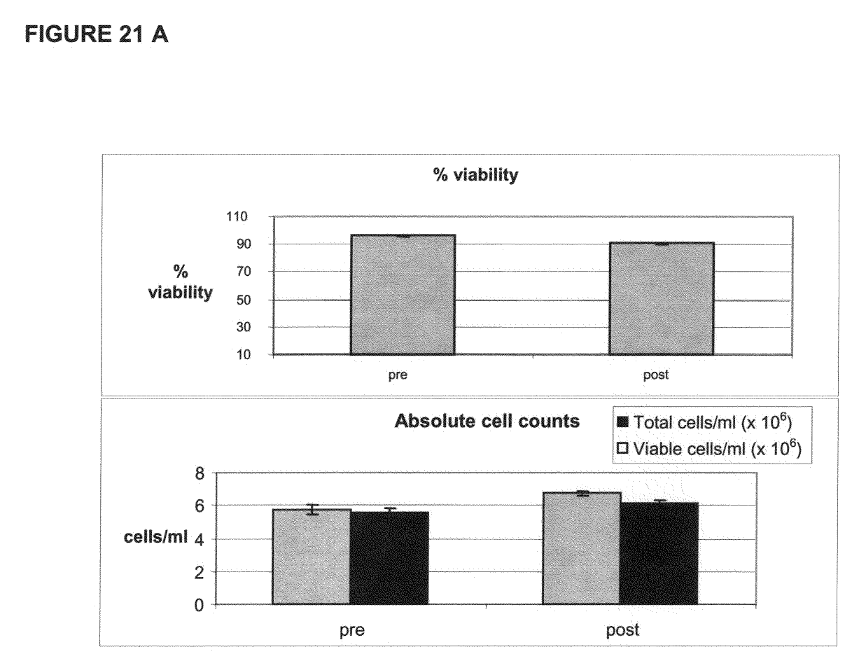

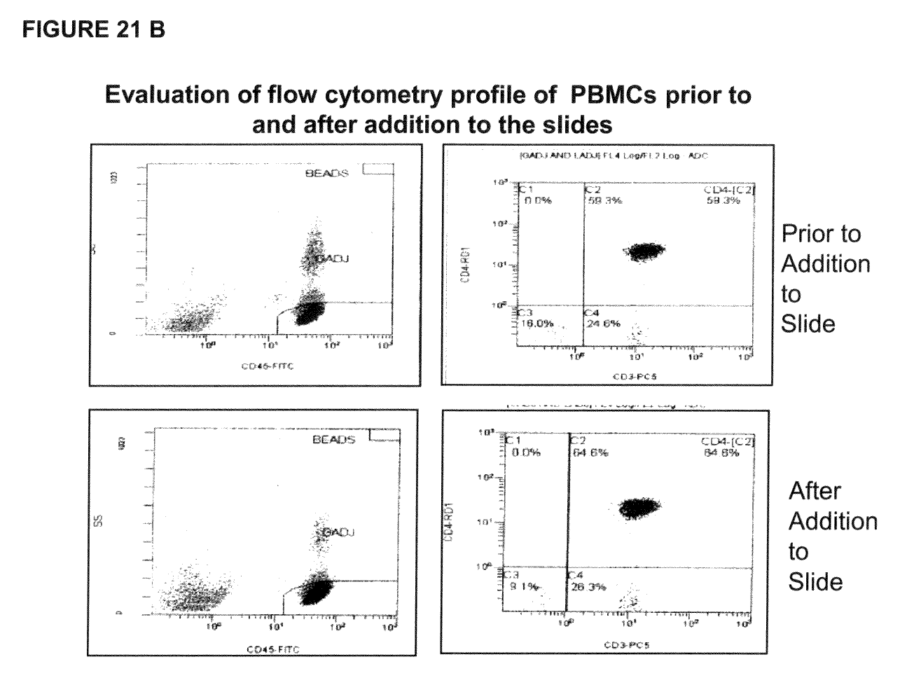

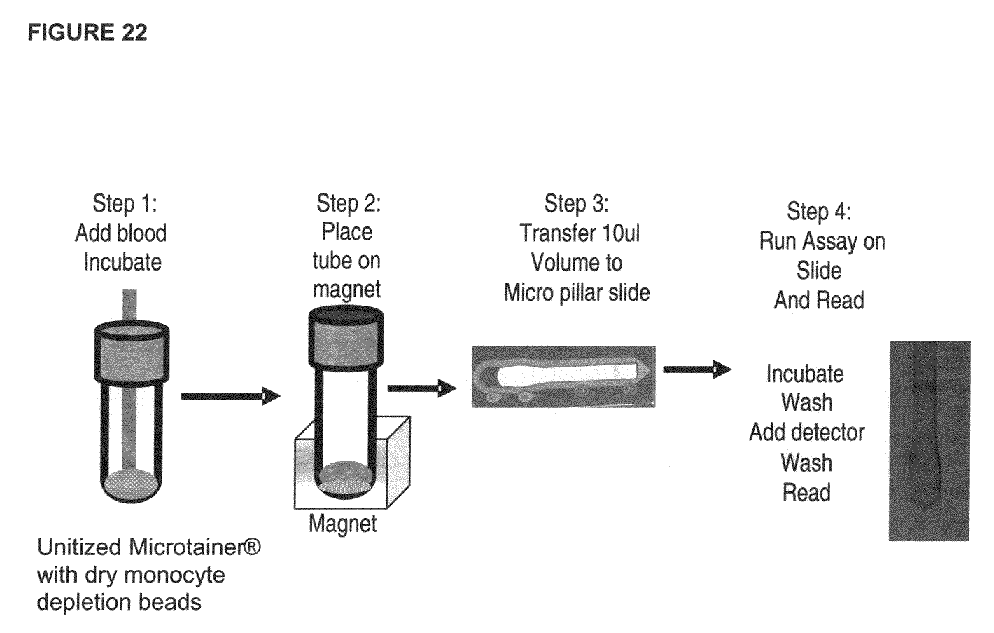

To evaluate the integrity and viability of cells after lateral flow on slides, ficol purified peripheral blood mononuclear cells (PBMCs) were run on micropillar slides using the assay conditions described in Example 6 (protocol for antibody capture of cells on chips using a humidified chamber) in the absence of capture antibody. In place of a wick at the waste zone the cells flowing through were collected by capillary action and evaluated on a Vi-Cell™ Cell Viability Analyzer (Beckman Coulter) for integrity and viability as well as by flow cytometry. Both the viability and phenotypic characteristics of the cells are maintained after lateral flow on the micropillar slides. See FIG. 21.

### Example 13

### Evaluation of Monocyte Depletion of Normal Whole Blood Samples Using CD14 Magnetic Beads

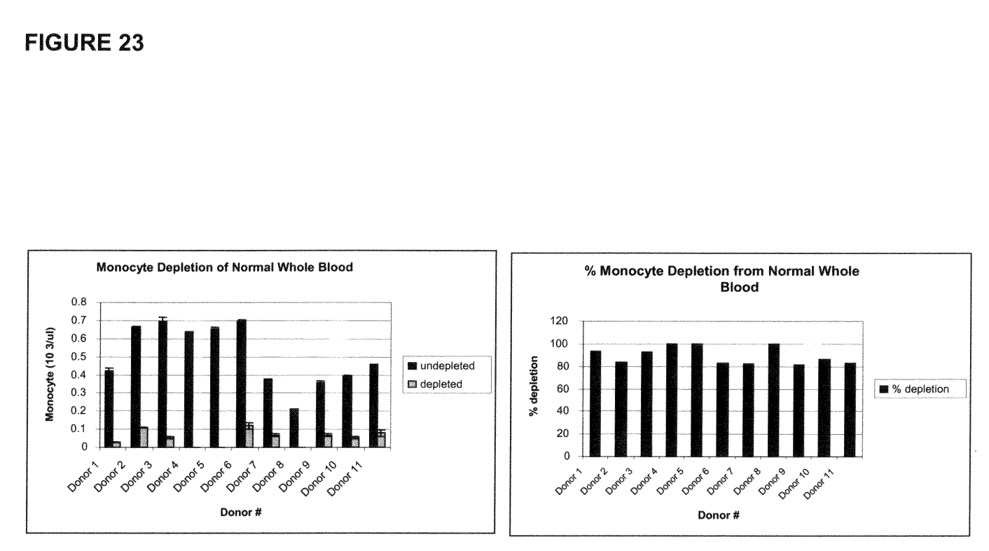

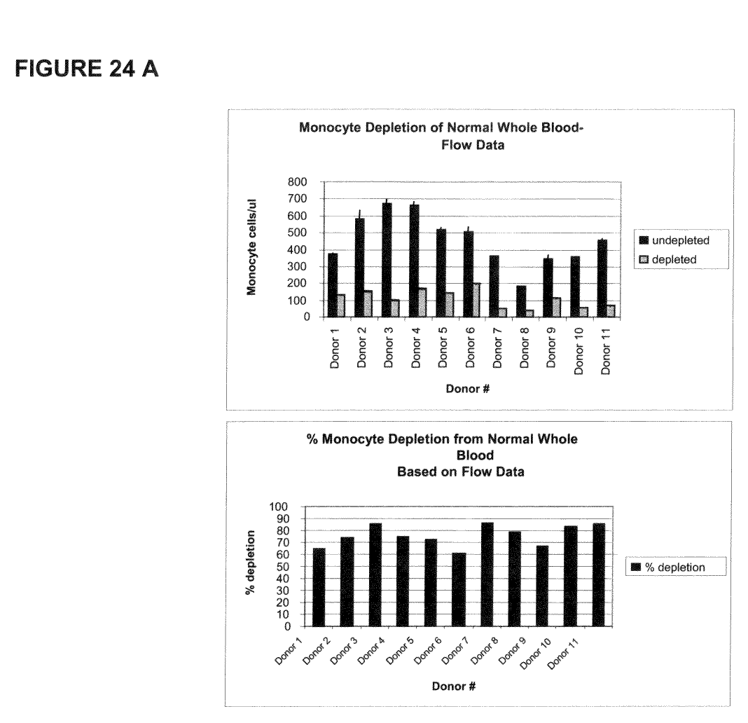

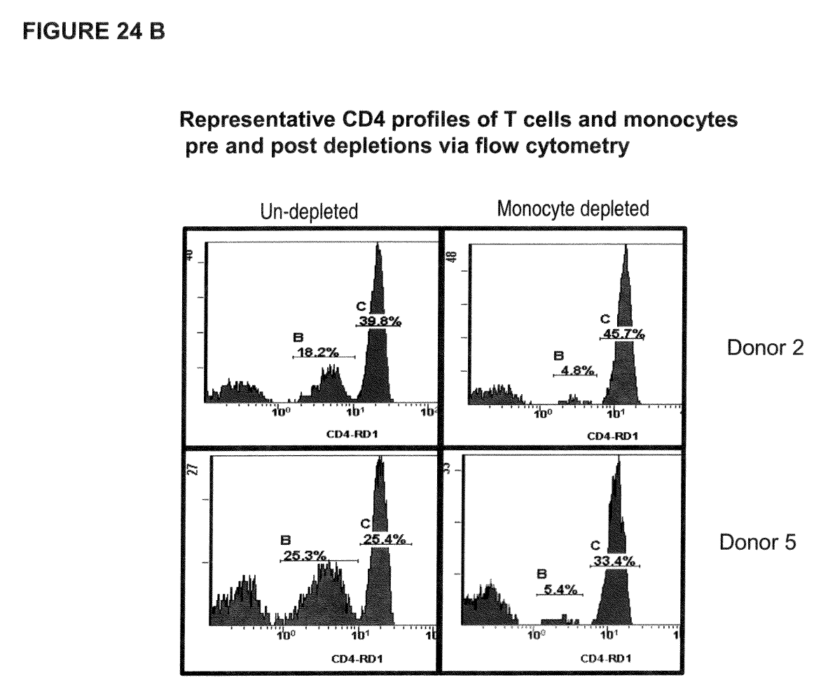

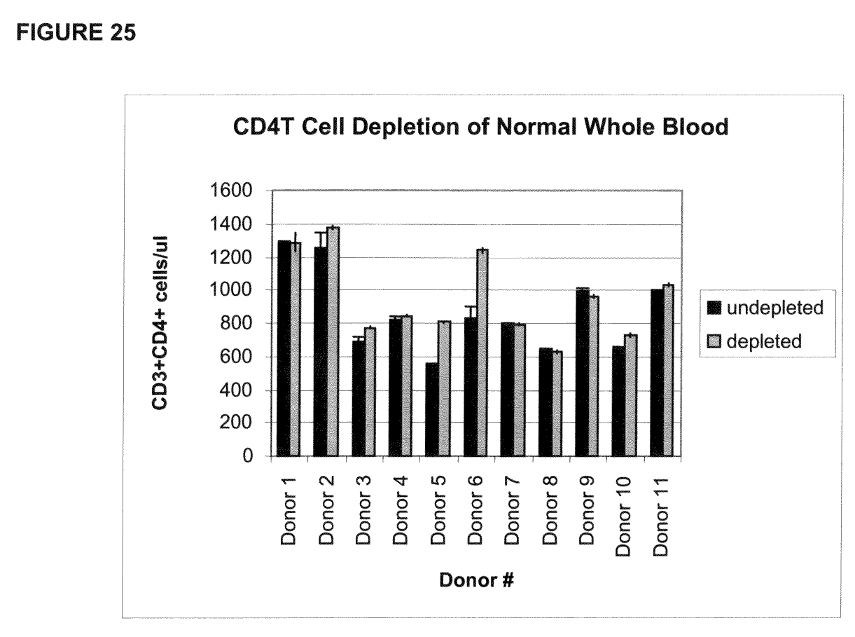

Whole blood samples from eleven normal donors was depleted of monocytes using “dry” magnetic monocyte depletion (CD14) beads (Beckman Coulter Inc). Blood was collected in a unitized Microtainer® (Becton Dickinson) and incubated with the magnetic CD14 beads for 10 minutes. The Microtainer® was subsequently placed on a magnet for two minutes. Depleted blood was analyzed on a Coulter® LH 750 Hematology Analyzer (see FIG. 23) and on a flow cytometer (see FIG. 24). For analysis by flow cytometry, samples were stained with Tetra 1 (4 color reagent-CD3/CD4/CD8/CD45) and absolute CD4+ monocyte counts were determined by gating on the lymph and monocyte populations via scatter and CD4+ phenotyping. CD4+CD3+ lymphocyte counts were also determined post-monocyte depletion by flow cytometry. See FIG. 25. Dry magnetic beads used to deplete monocytes prevent the dilution of whole blood while consistently enabling for at least 80% depletion of monocytes from the whole blood preparation.

### Example 14

### Evaluation of Normal Donor Whole Blood Samples on Micropillar Slides

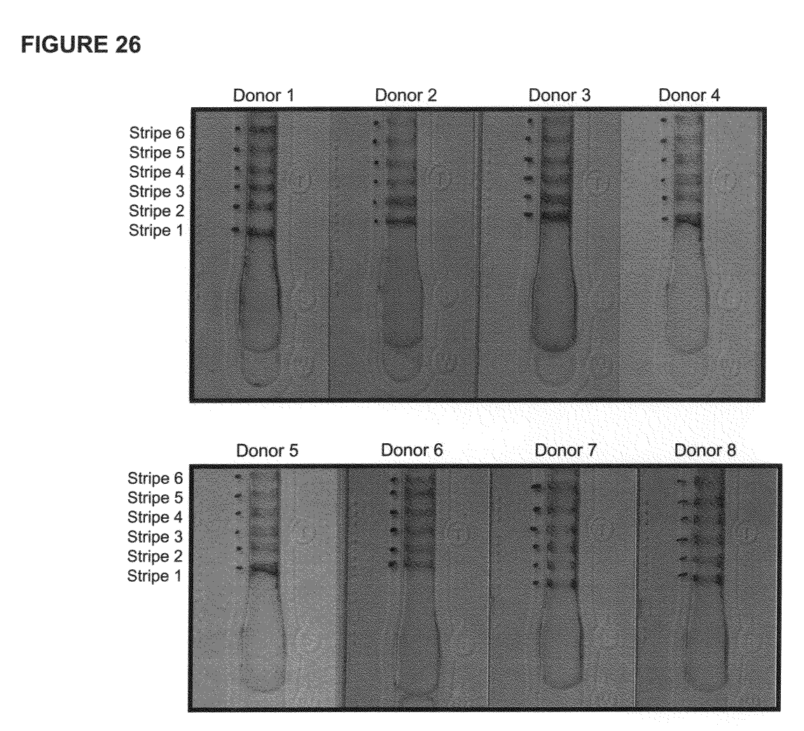

Whole blood samples from eight normal donors (CD4+ cell counts greater than 350 cells/μl) was depleted of monocytes using dry magnetic monocyte depletion (CD14) beads (Beckman Coulter Inc). Blood was collected in a unitized Microtainer® (Becton Dickinson) and incubated with the magnetic CD14 beads for 10 minutes. The Microtainer® was subsequently placed on a magnet for two minutes. Ten microliters of monocyte depleted blood was run on a micropillar slide with 6 stripes of T4 antibody (anti-CD4). Captured cells were visualized with anti-CD3 coupled black beads (Invitrogen). See FIG. 26. The assay should detect differences between 250 and 350 cells/μl such that stripe 6 is negative for 250 cells/μl and positive for 350 cells/μl. All donors had detectable bands on the 6th stripe indicating the presence of greater than 350 CD4+ T cells/μl in the monocyte depleted blood samples.

### Example 15

### Differentiation of 250CD4 T Cells/μl Versus 350 CD4 T Cells/μl

In order to demonstrate the differences between absolute cell counts, normal whole blood from 4 donors was depleted of CD4+ T cells and monocytes using CD4 magnetic beads. The use of “dry” magnetic beads prevented the dilution of the CD4 depleted whole blood sample. Separately, PBMCs and then CD4+ T-cells were purified from the same donor.

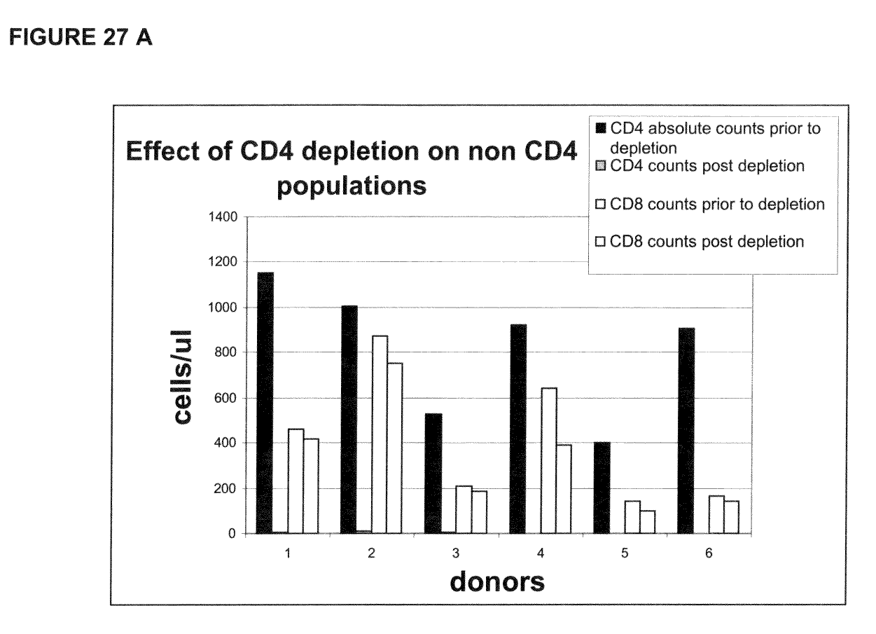

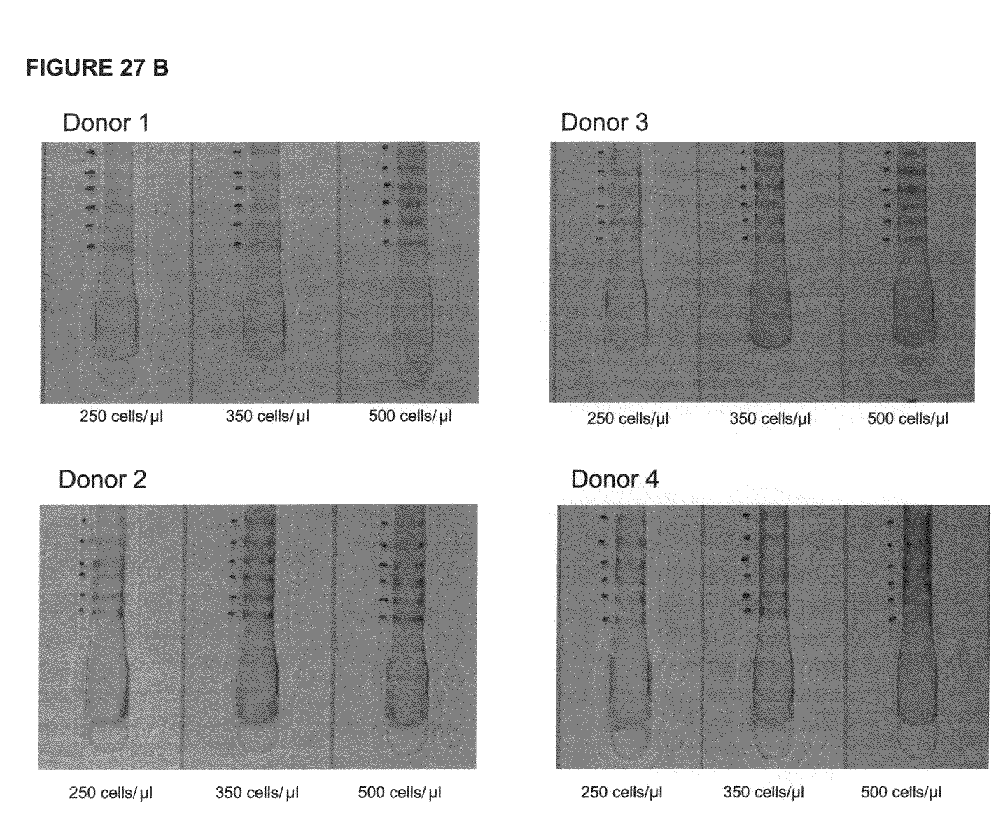

The CD4 depleted whole blood was then spiked with 250, 350 or 500 CD4+ T cells/μl. Each spiked preparation was then evaluated with tetra 1 reagent (CD3/CD4/CD8/CD45) to confirm absolute CD4+ T-cell count. In all cases the CD4+ T-cell spiked blood was within 20 cells/μl of expected concentration. Evaluation of the CD8+ cell counts before and after CD4+ cell depletion indicated that the CD8+ cell population was maintained. See FIG. 27A. Samples were then run on a 6-stripe anti-CD4 micropillar chip and cells were detected with black beads. See FIG. 27B. Differentiation of the intensity of the band in stripe 6 for the 250, 350 and 500 cells/μl samples was noted for Donors 1 and 3 and was noted to a degree for Donors 2 and 4. Thus, differentiation of absolute CD4+ cell counts in whole blood has been demonstrated in normal donors.

### Example 16

### Intra and Inter-Operator Variability

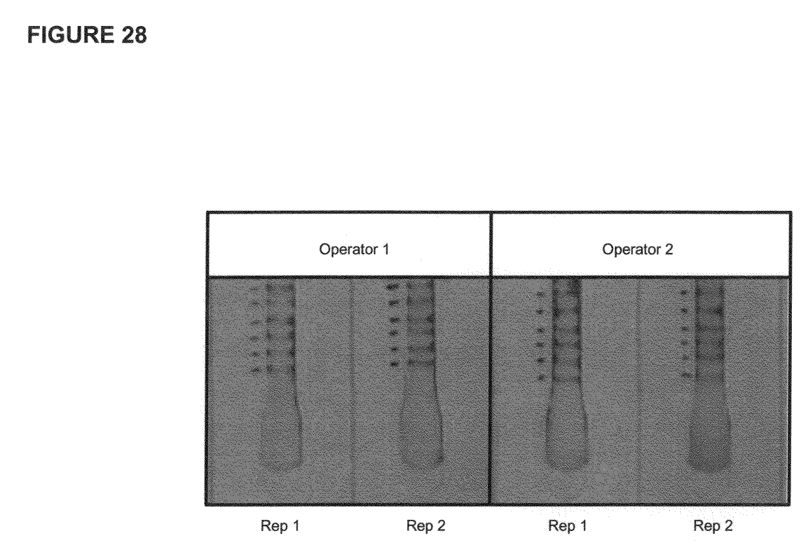

Intra and inter-operator variability of the present assay was evaluated. See FIG. 28. Intra-operator precision for operators 1 and 2, as well as inter-operator precision between operators 1 and 2, was visibly consistent. Thus, the assay of the invention is reliable and reproducible and can be used by operators with minimal training in resource-limited settings.

### Example 17

### Evaluation of HIV Donors on a Micropillar Slide

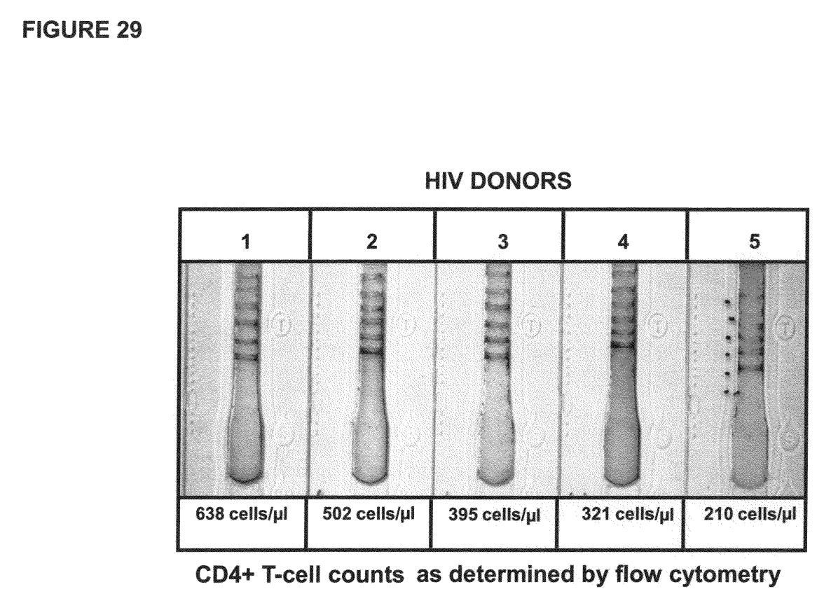

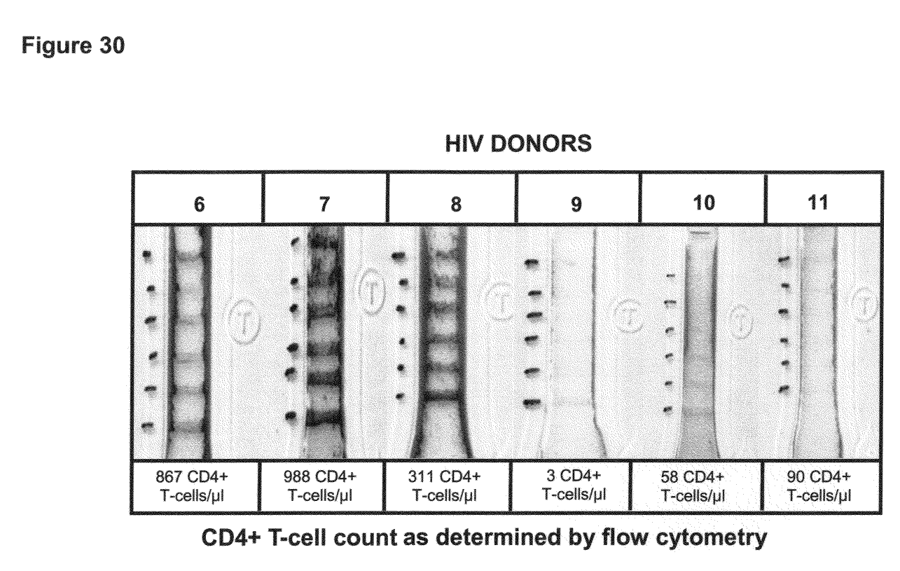

Whole blood samples from five HIV infected individuals were collected and CD4+ cell counts were determined by standard flow cytometry. Cell samples were then depleted of monocytes using CD14 magnetic beads as described above in Example 13. Monocyte depleted samples were run on 6 stripe anti-CD4 micropillar chips and captured cells were detected with CD3 black beads as described above. See FIGS. 29 and 30. The assay should detect the difference between 250 and 350 cells/μl: stripe 6 should be negative for 250 cells/μl and positive for 350 cells/μl. The results of this experiment agree with the CD4+ cell counts as determined by flow cytometry. Donors 5, 9, 10 and 11 with 210, 3, 58 and 90 CD4+ T-cells cells/μl, respectively, are negative for stripe 6.

### Example 18

### Evaluation of Onboard Depletion of Monocytes with ROM52 CD14 Antibody

To simplify the current assay and enable on board depletion of monocytes, the capture of monocytes by inclusion of a monocyte depletion zone on the chip prior to the CD4 capture zone was evaluated. ROM 52 CD14 antibody (Beckman Coulter Inc) was evaluated for capture of monocytes prior to and after the T4 zone. Whole blood, CD4-depleted whole blood, monocyte depleted whole blood, CD4 depleted whole blood spiked with CD4 T-cells, CD4 T cells alone, CD4 depleted whole blood spiked with monocytes, or monocytes alone were run on chips striped with before and after the T4 anti-CD4 detection zone. Cell binding was detected with anti-CD4 conjugated black beads.

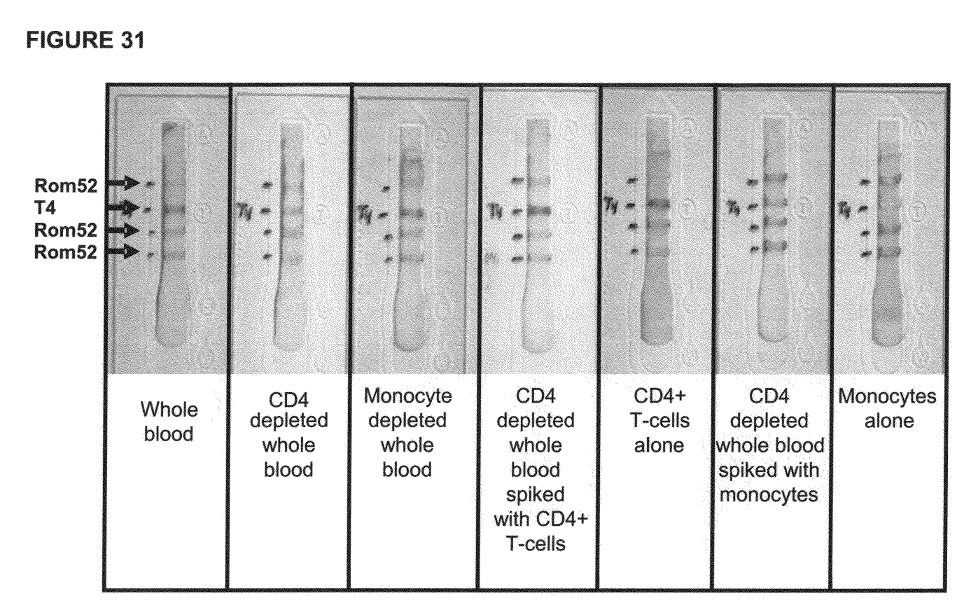

The majority of the binding to the T4 zone as detected by the comparison of whole blood, CD4 T cells alone or CD4 depleted whole blood spiked with CD4 T-cells appears to be CD4+ T-cell mediated. Purified monocytes or CD4 depleted whole blood spiked with monocytes showed higher binding to the ROM52 zone compared the T4 zone. See FIG. 31.

All publications and patents mentioned in the above specification are herein incorporated by reference. Various modifications and variations of the described method and device of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

What is claimed is:

1. A method for detecting a target cell in a sample, comprising the steps of:
(a) contacting a sample receiving zone of a device with a sample comprising intact cells, the device comprising one or more support materials capable of generating lateral flow of the sample, the one or more support materials comprising:
(i) the sample receiving zone for receiving the sample, and
(ii) one or more detection zones comprising an immobilized specific binding reagent, optionally covalently coupled to the support material, which binding reagent is capable
of forming a complex with an analyte on the target cell,
(b) flowing the sample across the one or more support materials from the sample receiving zone to the one or more detection zones, wherein the immobilized specific binding reagent forms a complex with the analyte on the target cell, and
(c) detecting the complex formed between the analyte on the target cell and the immobilized specific binding reagent.

2. The method of claim 1, wherein the one or more support materials is non-porous and comprises a multiplicity of projections perpendicular to a support surface, said projections having a height, a diameter, and a distance between the projections capable of generating lateral flow of the sample comprising intact cells.

3. The method of claim 2, wherein the projections have a diameter of about 10 μm to about 160 μm.

4. The method of claim 2, wherein the projections have a height of about 50 μm to about 150 μm.

5. The method of claim 2, wherein the projections have a distance between the projections of about 20 μm to about 200 μm.

6. The method of claim 2, wherein the projections have a diameter of about 45 μm to about 55 μm, a height of about 58 μm to about 72 μm, and a distance between the projections of about 27 μm to about 33 μm.

7. The method of claim 2, wherein the horizontal cross-sections of substantially all of the projections are either oval in shape, star shaped, circular, or rectangularly shaped.

8. The method of claim 2, wherein the non-porous support material comprises cyclo-olefin polymers, silicon, metal, plastic, polystyrene, polypropylene or glass and chemically activated to enable covalent coupling of immobilized reagent to the non-porous support.

9. The method of claim 1, wherein the detecting comprises
(a) labeling the target cells with a target cell specific binding reagent coupled to a detectable label, and
(b) detecting the detectable label.

10. The method of claim 9, wherein the target cell specific binding reagent forms a complex with an antigen on the target cell, and wherein the antigen is selected from the group consisting of CD3 and CD4.

11. The method of claim 9, wherein the target cells are labeled after application to the device.

12. The method of claim 9, wherein the detectable label is a liposome, a latex bead, a colloidal gold particle, and/or a colloidal silver particle conjugate.

13. The method of claim 1, wherein the device further comprises a control zone comprising an immobilized control specific binding reagent.

14. The method of claim 9, wherein the intensity of the label in the one or more detection zones correlates with the number of target cells in the sample.

15. The method of claim 9, wherein the one or more detection zones comprise a first detection zone and a second detection zone, wherein detection of the detectable label in the first detection zone indicates the target cell is present in the sample at a concentration within a first concentration range, and detection of the detectable label in the second detection zone indicates the target cell is present in the sample at a concentration within a second concentration range.

16. The method of claim 15, wherein the first concentration range is between about 200 to about 250 cells/μL, and wherein the second concentration range is about 350 cells/μL or higher.

17. The method of claim 1, wherein the sample is whole blood.

18. The method of claim 1, wherein the target cell is a CD4 lymphocyte and the analyte is a CD4 antigen.

19. The method of claim 17, wherein the whole blood sample is depleted of monocytes before application to the device.

20. The method of claim 17, wherein the device further comprises a monocyte depletion zone comprising an immobilized monocyte specific binding reagent, and wherein the monocyte depletion zone is arranged in the one or more support materials such that the sample flows sequentially through the sample receiving zone, the monocyte depletion zone, and finally the detection zone.

21. The method of claim 20, wherein the immobilized monocyte specific binding reagent forms a complex with CD14.

22. A kit for detecting CD4 lymphocytes in a whole blood sample, comprising:
(a) labeled CD3 and/or CD4 specific binding reagent; and
(b) a device comprising one or more non-porous support materials capable of generating lateral flow of intact cells, an immobilized CD4 specific binding reagent, and an immobilized control cell specific binding reagent, and wherein the device does not comprise a material capable of nonspecifically trapping the intact cells positioned upstream of the detection zone.

23. The kit of claim 22, wherein the diagnostic device further comprises:
(a) a sample receiving zone for receiving the whole blood sample; and
(b) a first detection zone comprising the immobilized CD4 specific binding reagent, a second detection zone comprising the immobilized CD4 specific binding reagent, and a third detection zone comprising the immobilized control cell specific binding reagent.

24. A device for detecting a target cell in a sample comprising intact cells, the device comprising one or more support materials capable of generating lateral flow of the sample, the one or more support materials comprising
(i) a sample receiving zone for receiving the sample comprising intact cells, wherein the device does not comprise a material capable of nonspecifically trapping the intact cells positioned upstream of the detection zone, and
(ii) one or more detection zones comprising an immobilized specific binding reagent capable of forming a complex with an analyte on the target cell.

25. The device of claim 24, wherein the one or more support materials is non-porous and comprises a multiplicity of projections substantially perpendicular to a support surface, the projections having a height, a diameter, and a distance between the projections capable of generating lateral flow of the sample comprising intact cells.

26. The device of claim 25, wherein the projections have a diameter of about 45 μm to about 55 μm, a height of about 58 μm to about 72 μm, and a distance between the projections of about 27 μm to about 33 μm.

27. The device of claim 24, wherein the device further comprises a monocyte depletion zone comprising an immobilized monocyte specific binding reagent.

28. The device of claim 24, wherein the one or more detection zones comprise a first detection zone and a second detection zone, wherein detection of a complex formed between the analyte on the target cell and the immobilized specific binding reagent in the first detection zone indicates the target cell is present in the sample at a concentration within a first concentration range, and detection of a complex formed between the analyte on the target cell and the immobilized specific binding reagent in the second detection zone indicates the target cell is present in the sample at a concentration within a second concentration range.

29. The device of claim 24, wherein the device further comprises a cell depletion zone comprising an immobilized specific binding reagent that forms a complex with a specific subpopulation of cells.

30. A method for detecting a target cell in a sample, comprising
(a) contacting a sample receiving zone of a device with a sample comprising intact cells, the device comprising one or more porous support materials capable of allowing radial diffusion of the sample, the one or more support materials comprising
(i) the sample receiving zone for receiving the sample, and
(ii) one or more detection zones comprising an immobilized specific binding reagent capable of forming a complex with an analyte on the target cell,
(b) flowing the sample by radial diffusion across the one or more support materials from the sample receiving zone to the detection zone, wherein the immobilized specific binding reagent forms a complex with the analyte on the target cell, and
(c) detecting the complex formed between the analyte on the target cell and the immobilized specific binding reagent.
